SEQUENCE LISTING (1 sequence)

SEQ ID NO: 1 - DNA, 34 bp, Homo sapiens
  source 1..34: mol_type = unassigned DNA
tgggccggggacccgggaggggtcgggacggggc

(19) United States
(12) Patent Application Publication
(10) Pub. No.: US 2024/0331873 A1
(43) Pub. Date: Oct. 3, 2024

(54) VARIANT BASED DISEASE DIAGNOSTICS AND TRACKING

(71) Applicant: GRAIL, LLC, Menlo Park, CA (US)

(72) Inventor: Oliver Claude Venn, San Francisco, CA (US)

(21) Appl. No.: 18/627,253

(22) Filed: Apr. 4, 2024

Related U.S. Application Data
(63) Continuation of application No. 15/411,879, filed on Jan. 20, 2017, now abandoned.
(60) Provisional application No. 62/286,103, filed on Jan. 22, 2016.

(51) Int. Cl. G16H 50/30; C12Q 1/6886; G16B 20/20; G16B 25/00; G16B 25/10; G16B 30/00

(52) U.S. Cl. CPC G16H 50/30; C12Q 1/6886; G16B 20/20; G16B 25/00; G16B 25/10; G16B 30/00; C12Q 2600/106; C12Q 2600/118; C12Q 2600/156

(57) ABSTRACT

Aspects of the invention relate to methods for tracking patient health by longitudinally tracking genetic variants in patients, such that it is possible to provide a tumor, or mutation, classification signature. Longitudinal tracking improves the ability to detect minimal residual disease (MRD; the small number of cells that remain in the patient after treatment and/or during remission) and/or treatment response at an early stage, both of which can help guide treatment decisions and guard against missing different intra-/inter-tumor responses in a patient.

### CROSS REFERENCE TO RELATED APPLICATIONS

This application is a continuation of U.S. application Ser. No. 15/411,879, filed Jan. 20, 2017, which claims the benefit of the filing date under 35 USC § 119(e) of U.S. Provisional Patent Application No. 62/286,103, filed on Jan. 22, 2016. The disclosures of which application are herein incorporated by reference in their entireties.

The instant application contains a sequence listing which is being submitted electronically in XML format and is hereby incorporated by reference in its entirety. Said XML copy, created on Apr. 29, 2024, is named “60793.00045US02 (GRA-0004-C1-US); ST.26 Sequence Listing” and is 2 kilobytes in size.

### FIELD OF THE INVENTION

Aspects of the invention relate to methods for tracking patient health using patient mutation signatures and telomere specific tandem repeat sequences.

### BACKGROUND

Cancer is a devastating disease affecting millions of individuals every year. The disease is characterized by a complex lineage of genomic alterations, or mutations, manifesting as intra- and inter-tumor genetic heterogeneity. See, e.g., Knudson Proc Natl Acad Sci, 68: 820-823 (1971); Gerlinger et al., N Engl J Med, 366: 883-892 (2012); Campbell et al., Proc Natl Acad Sci, 105: 13081-13086 (2008); Robbins et al., Nature Medicine, 19: 747-752 (2013); Murtaza et al., Nature Communications, 6:8760 (November 2015); and Hong et al., Nature Communications 6:6605 (April 2015).

Some alterations are causal and drive tumor progression, while other events have little functional consequence and are known as passenger mutations. The accumulation of alterations is observed as genetic heterogeneity within a tumor and/or between tumors in individual patients and between patients. An example of this can be seen in FIG. 1, wherein the lineage of an initiating tumor cell is shown. The ancestral cell arises at time t0 and genetically distinct sub-populations (subclones) arise during cell division adding new branches to the tree. The relative population size of each subclone is represented by the width of each branch. Over time three subclones are generated S(0,1), S(0,2), and S(0,3), each distinguished by its own set of somatic alterations. If no reversion mutations occur and there is no recombination, the mutations can be represented as a nested tree object (e.g. S(0,1) contained in S(0,3)). A metastasis S(3,0) is derived from rapidly expanding subclone S(3,0). In this particular example, the number of cells in S(0,2) decreases, the number of cell in S(0,1) remains stable, and the number of cells in S(0,3) increases.

However, somatic genetic heterogeneity generates two challenges in tumor classification: tumors undergo rapid evolution through time and, despite arising in the same tissue in two+ individuals, the tumors can be genetically distinct with different prognosis and treatment response.

Genetic tests that focus on single loci, such as the KRAS mutation status test, have demonstrated utility in therapy choice, such as informing the decision whether to use tyrosine kinase inhibitors. See, e.g., Plesec et al., Adv. Anal Pathol. (2009). However, single locus tests are inadequate to capture the genetic heterogeneity in cancer and therefore have limited utility in classification. Some studies have assessed heterogeneity using multi-region sequencing at one point in time, while others have tracked pre-defined mutations through time. Accordingly, there is a need to develop a method to create a tumor classification signature from sampling part or all of the genetic variation in a patient through time.

### SUMMARY

Aspects of the invention relate to methods for tracking patient health by longitudinally tracking genetic variants in patients, such that it is possible to provide a tumor, or mutation, classification signature. Longitudinal tracking improves the ability to detect minimal residual disease (MRD; the small number of cells that remain in the patient after treatment and/or during remission) and/or treatment response at an early stage, both of which can help guide treatment decisions and guard against missing different intra-/inter-tumor responses in a patient. Systems and methods of the invention relate to identifying and tracking the genetic diversity in individual tumors and/or patients in order to predict and understand treatment resistance and to generate neo-antigens that can be targets of host immune response. These alterations represent a discriminating and fundamental signature of the tumor that can ultimately be used to classify the tumor and predict progression and treatment efficacy.

In accordance with methods of the invention, mutation signatures can be created from sampling part or all of the genetic variation in a patient through time. These longitudinal signatures can then be used to classify patient status against one or more databases of known healthy and sick individuals' signatures. As each additional patient's signature and health status is refined overtime, the next patient benefits from the improved discriminatory power of the classification database.

According to one embodiment of the invention, the health status of a patient can be tracked by creating a mutation signature for a patient. The mutation signature is determined from a number of variables including a total number of observed variants in a nucleic acid sample of the patient, a sequence context factor for each of the observed variants, allele frequency of each of the observed variants, nucleic acid polymer fragment size, inferred DNA replication timing, chromatin structure (e.g., open v. closed chromatin structure), DNA methylation status, inter-mutation distance, predicted functional consequence of mutations, estimates of selection (e.g., the ratio of non-synonymous to synonymous mutations in a patient), and variant type classification. The mutation signature for the patient is then compared to a reference database containing mutation signatures of patients with known health statuses, wherein a diagnosis or therapy can be determined for the patient. Variant type classifications can include telomeric sequence copy number variation, chromosomal instability, translocation, inversion, insertion, deletion, loss of heterozygosity, amplification, kataegis, and microsatellite instability.

In one aspect of the invention, a longitudinal mutation signature can be determined for the patient by comparing a plurality of mutation signatures for the patient over time to a reference database, wherein the reference database also contains longitudinal mutation signatures of patients with known health statuses, before determining a diagnosis or therapy. In some embodiments, a longitudinal mutation signature comprises a first mutation signature for the patient from a first time point, and a second mutation signature for the patient from a second time point. In some embodiments, the first time point is before a treatment and the second time point is after the treatment. In some embodiments, the treatment comprises a tumor resection surgery. In some embodiments, the treatment comprises administration of an anti-cancer therapeutic agent.

In another aspect of the invention, a health status for the patient is obtained and added to the database along with the mutation signature of the patient. Information from the patient, such as age, gender, race, ethnicity, family disease history (e.g., the presence of Lynch syndrome, inherited BRCA 1/2 mutations, etc.), weight, body mass index, height, prior and/or concurrent infections, environmental exposures, and smoking history can be obtained and also compared to the one or more databases of patients with known health statuses. Furthermore, gene product levels, such as protein biomarker levels, can also be obtained from the patient and compared to levels of patients with known health statuses in the one or more databases of patients with known health statuses.

In order to determine the mutation signature from the nucleic acids of a patient, a sample can be obtained from the patient. The sample can comprise, e.g., a tissue sample, a body fluid, a cell sample, or a stool sample. In certain embodiments, a sample comprises a body fluid, such as whole blood, saliva, tears, sweat, sputum, or urine. In some embodiments, only a portion of the whole blood, such as blood plasma or cell free nucleic acid is used. In other embodiments, the sample is a tissue sample, such as a formalin-fixed paraffin-embedded (FFPE) tissue sample, a fresh frozen (FF) tissue sample, or a combination thereof.

Methods of the invention can also be used to determine intra-tumor or inter-tumor heterogeneity from observed variants over time. Furthermore, treatment efficacy can also be determined by monitoring observed variants over time before and after treatment of the patient. In this manner, the patient can be monitored for minimal residual disease.

In another embodiment, patient health can be tracked by performing an assay on nucleic acid obtained from a patient to determine telomere specific tandem repeat sequences, creating a telomere integrity score comprising a frequency distribution of telomere tandem repeats, producing a longitudinal trajectory of the telomere integrity score of nucleic acid obtained from the patient at two or more time points, comparing the longitudinal trajectory to a reference database containing longitudinal trajectories of patients with known health statuses, and determining a diagnosis or therapy for the patient.

In one aspect of the invention, the cell free nucleic acid is obtained from a body fluid, such as whole blood, saliva, tears, sweat, sputum, and urine. When the body fluid is whole blood, a portion of the whole blood, such as plasma can be used.

In another aspect of the invention, a health status for the patient is obtained and added to the database along with the longitudinal trajectory of the patient. Information from the patient, such as age, gender, race, ethnicity, family disease history, weight, body mass index, height, prior and/or concurrent infections, environmental exposures, and smoking history can be obtained and also compared to the one or more databases of patients with known health statuses. Gene product levels, such as protein biomarker levels, can also be obtained from the patient and compared to levels of patients with known health statuses in the one or more databases of patients with known health statuses. Furthermore, a TERT promoter mutation profile can be obtained from the patient and compared to TERT promoter mutation profiles in one or more databases of patients with known health statuses.

The frequency distribution of telomere tandem repeats can also be normalized. This can be done by comparing the frequency distribution to a control sequence having the same proportions of individual nucleobases as the telomere specific tandem repeat sequences. The frequency distribution can also be normalized by comparing the frequency distribution of telomere tandem repeats to a reference database of frequency distributions.

In one aspect of the invention, the assay can be sequencing, such as whole genome sequencing. The sequencing can also be targeted sequencing such as targeted PCR amplification or hybrid capture using selectable oligonucleotides.

In another aspect, the telomere specific tandem repeat sequences can be identified through alignment to a telomeric reference sequence or analysis of k-mer frequencies.

### BRIEF DESCRIPTION OF THE DRAWINGS

FIG. 1 shows the lineage of an initiating tumor cell through time.

FIG. 2 is a chart depicting the depth of sequence coverage of whole genome sequencing (WGS) from cfDNA versus tissue biopsy.

FIG. 3 is a chart depicting whole genome sequencing (WGS) identified mutations stratified by triplet sequence context from a metastatic melanoma cancer patient (PT0001). The first and second panels show the identified mutations at a first time point and a second time point, respectively. The second time point was taken after multiple therapy regimens. The third panel shows the relative change in frequency between time points.

FIG. 4 is a chart showing the allele frequency of validated tumor mutations in a thoracic cancer patient before and after resection surgery.

FIGS. 5A-K are charts showing the allele frequency of 100 somatic mutations in protein coding regions over a treatment period in a metastatic melanoma cancer patient.

FIG. 6 is a flow chart depicting a method in accordance with an embodiment of the invention.

FIG. 7 is a chart showing the empirical distribution of the number of whole genome sequencing reads from cfDNA containing repeated telomeric sequences from a melanoma cancer patient PT0001.

FIG. 8 is a diagram of a system in accordance with embodiments of the invention.

FIG. 9 is a graph showing somatic variant allele frequencies measured in a colorectal cancer (CRC) patient before and after surgical tumor excision.

FIG. 10 is a graph showing somatic variant allele frequencies measured in a CRC patient before and after surgical tumor excision.

FIG. 11 is a graph showing somatic variant allele frequencies measured in a CRC patient before and after surgical tumor excision. The tree on the right hand side represents a potential underlying lineage of cancer cells in the patient; the tree is consistent with allele frequency trajectories under surgery.

FIG. 12 is a collection of bar graphs that show allele frequencies of microsatellite repeats from cfDNA sequencing from different patients.

FIG. 13 is a collection of bar graphs that show allele frequencies of microsatellite repeats from cfDNA and genomic DNA sequencing for various sample types including cancer patients and synthetic controls using WGS and targeted sequencing.

FIG. 14 is a collection of bioanalyzer traces that show fragment size of extracted cfDNA in base pairs.

FIG. 15 is a collection of bioanalyzer traces that show cfDNA library fragment size in base pairs prior to PCR amplification.

FIG. 16 is a collection of bioanalyzer traces that show cfDNA library fragment size in base pairs after 8 cycles of PCR amplification.

FIG. 17 is a collection of bioanalyzer traces that show cfDNA library fragment size in base pairs after 12 cycles of PCR and clean-up.

FIG. 18 is a time-course representation of a time course of disease progression for the patient, and shows treatment, observations and sample collection time points.

FIG. 19A is a panel of pileup views of sequencing reads from PT0001 at the core promoter of telomerase reverse transcriptase (TERT). Four panels (top to bottom): white blood cell derived genomic DNA sequencing, cfDNA sequencing at time point 1, cfDNA time point 2 sequencing, and tumor biopsy sequencing. Between dashed vertical lines A letters represent the reverse-complement of mutant alleles copies of a known activating C>T mutation.

FIG. 19B is a table that summarizes the data in FIG. 19A, showing the read counts at chr5: 129,250 for the indicated samples.

FIGS. 20A-C provide summary tables of colorectal cancer patient information and predicted disease recurrence from cfDNA analysis.

### DETAILED DESCRIPTION

Methods of the invention involve longitudinally tracking multiple somatic alterations, such that it may be possible to guard against missing different intra-/inter-tumor responses in a patient and improve the ability to detect minimal residual disease and/or treatment response. This can be accomplished through the creation of a mutation signature or signatures and/or the creation of a telomere integrity score determined from nucleic acid obtained from a patient, both of which can be longitudinally tracked.

The methods initially involve obtaining a sample, e.g., a tissue or body fluid that is suspected to include a cancer-associated gene or gene product. The sample may be collected in any clinically acceptable manner. A tissue is a mass of connected cells and/or extracellular matrix material, e.g., skin tissue, hair, nails, endometrial tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues. The tissue can be prepared and provided as any one of the tissue samples types known in the art, such as, for example and not limitation, formalin-fixed paraffin-embedded (FFPE) and fresh frozen (FF) tissue samples.

A body fluid is a liquid material derived from, for example, a human or other mammal. Such body fluids include, but are not limited to, mucous, blood, plasma, serum, serum derivatives, bile, maternal blood, phlegm, saliva, sweat, tears, sputum, amniotic fluid, menstrual fluid, urine, and cerebrospinal fluid (CSF), such as lumbar or ventricular CSF. A sample may also be a fine needle aspirate or biopsied tissue. A sample also may be media containing cells or biological material. A sample may also be a blood clot, for example, a blood clot that has been obtained from whole blood after the serum has been removed. A sample may also be stool. In certain embodiments, the sample is drawn whole blood. In one aspect, only a portion of whole blood is used, such as plasma, red blood cells, white blood cells, and platelets.

The sample can include nucleic acid not only from the subject from which the sample was taken, but also from other species such as viral DNA/RNA. Nucleic acid can be extracted from the sample according to methods known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, cell free nucleic acid is extracted from the sample.

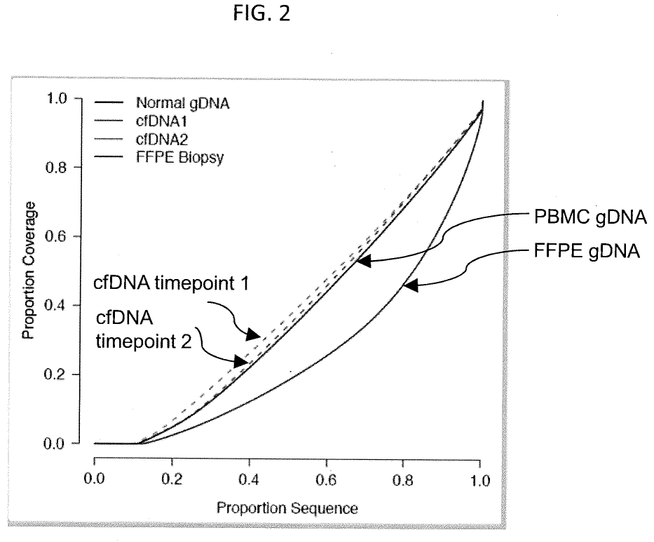

In some embodiments, cell free DNA (cfDNA) is extracted from the sample. Cell free DNA are short base nuclear-derived DNA fragments present in several bodily fluids (e.g. plasma, stool, urine). See, e.g., Mouliere and Rosenfeld, PNAS 112(11): 3178-3179 (March 2015); Jiang et al., PNAS (March 2015); and Mouliere et al., Mol Oncol, 8(5):927-41 (2014). Tumor derived circulating tumor DNA (ctDNA) constitutes a minority population of cfDNA, in some embodiments, varying up to about 50%. In some embodiments, ctDNA varies depending on tumor stage and tumor type. In some embodiments, ctDNA varies from about 0.001% up to about 30%, such as about 0.01% up to about 20%, such as about 0.01% up to about 10%. The covariates of ctDNA are not fully understood, but appear to be positively correlated with tumor type, tumor size, and tumor stage. E.g., Bettegowda et al, Sci Trans Med, 2014; Newmann et al, Nat Med, 2014. Despite the challenges associated with the low population of ctDNA in cfDNA, tumor variants have been identified in ctDNA across a wide span of cancers. E.g., Bettegowda et al, Sci Trans Med, 2014. Furthermore, analysis of cfDNA versus tumor biopsy is less invasive and methods for analyzing, such as sequencing, enable the identification of sub-clonal heterogeneity. Analysis of cfDNA also provides for more uniform genome-wide sequencing coverage than with a tissue tumor biopsy, as shown in FIG. 2.

An exemplary procedure for preparing nucleic acid from blood follows. Blood may be collected in 10 ml EDTA tubes (available, for example, from Becton Dickinson). Streck cfDNA tubes (Streck, Inc., Omaha, Nebraska) can be used to minimize contamination through chemical fixation of nucleated cells but little contamination from genomic DNA is observed when samples are processed within 2 hours or less as in some embodiments. Beginning with a blood sample, plasma may be extracted by centrifugation at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing as plasma may be more stable than storing extracted cfDNA.

Plasma DNA can be extracted using any suitable technique. For example, in some embodiments, plasma DNA can be extracted using one or more commercially available assays, for example, the Qiagen QIAmp Circulating Nucleic Acid kit (Qiagen N.V., Venlo Netherlands). In certain embodiments, the following modified elution strategy may be used. DNA may be extracted using the Qiagen QIAmp circulating nucleic acid kit following the manufacturer's instructions (maximum amount of plasma allowed per column is 5 ml). If cfDNA is being extracted from plasma where the blood was collected in Streck tubes, the reaction time with proteinase K may be doubled from 30 min to 60 min. Preferably, as large a volume as possible should be used (i.e., 5 mL). In various embodiments, a two-step elution may be used to maximize cfDNA yield. First, DNA can be eluted using 30 μl of buffer AVE for each column. A minimal amount of buffer necessary to completely cover the membrane can be used in elution in order to increase cfDNA concentration. By decreasing dilution with a small amount of buffer, downstream desiccation of samples can be avoided to prevent melting of double stranded DNA or material loss. Subsequently, about 30 μl of buffer for each column can be eluted. In some embodiments, a second elution may be used to increase DNA yield.

In certain embodiments, a genomic sample is collected from a subject followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, for example, Lapidus (U.S. Pat. No. 7,666,593), the content of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014).

Isolation of nucleic acid from a sample in accordance with the methods of the invention can be done according to any method known in the art. For example, RNA may be isolated from eukaryotic cells by procedures that involve lysis of the cells and denaturation of the proteins contained therein. Tissue of interest includes gametic cells, gonadal tissue, endometrial tissue, fertilized embryos, and placenta. RNA may be isolated from fluids of interest by procedures that involve denaturation of the proteins contained therein. Fluids of interest include those fluids listed above. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

Once the nucleic acid has been extracted, it can be assayed to determine genetic variants. The terms “variants”, “variations”, and “mutations” as used interchangeably herein refer to genetic sequences that are different from a wild type or control sequence. Any assay known in the art may be used to determine presence or absence of a genetic variation. Conventional methods can be used, such as those employed to make and use nucleic acid arrays, amplification primers, hybridization probes, and can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, CA), Applied Biosystems (Foster City, CA), and Agilent Technologies (Santa Clara, CA).

In some embodiments of the invention, nucleic acids are sequenced in order to detect variants (i.e., mutations) in the nucleic acid. The nucleic acid can include a plurality of nucleic acids derived from a plurality of genetic elements. Methods of detecting sequence variants are known in the art, and sequence variants can be detected by any sequencing method known in the art, e.g., ensemble sequencing (wherein consensus sequencing is conducted by integrating sequencing/PCR errors across PCR duplicates) or single molecule sequencing.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Methods have also been developed based upon sequencing by hybridization. See, e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109). Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslavsky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety.

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. Yet another example of a sequencing technique that can be used in the methods of the provided invention is nanopore sequencing (Soni G V and Meller A. (2007) Clin Chem 53: 1996-2001). Another example of a sequencing technique that can be used in the methods of the provided invention involves using a chemical-sensitive field effect transistor (chemFET) array to sequence DNA (for example, as described in US Patent Application Publication No. 20090026082). Another example of a sequencing technique that can be used in the methods of the provided invention involves using a electron microscope (Moudrianakis E. N. and Beer M. Proc Natl Acad Sci USA. 1965 March; 53:564-71).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety).

While the combination of the potential sequence of genetic variants and their sequence of occurrence, in addition to their relative frequency allows for essentially infinite combinations, the creation of a mutation signature is made practical by establishing a framework within which to classify the variants.

In some embodiments, variations are measured at a single point in time to determine a mutation signature for a patient. In some embodiments, variations are longitudinally tracked over time to facilitate the generation of a longitudinal mutation signature for a patient. For example, in some embodiments, two or more samples can be collected from a patient over time, and the collected samples can be used to generate a longitudinal mutation signature for the patient. In some embodiments, a first sample is collected at a first time point and a second sample is collected at a second time point. Research has demonstrated that cfDNA can have a clearance time ranging from about 15 mins up to several hours, depending on the rate of clearance (Forte V A, et al., The potential for liquid biopsies in the precision medical treatment of breast cancer, Cancer Biology & Medicine. 2016; 13(1):19-40. doi:10.28092/j.issn.2095-3941.2016.0007. Accordingly, in some embodiments, the first and second time points are separated by an amount of time that ranges from about 15 minutes up to about 25 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5 or about 25 years.

In some embodiments, the first time point is before the inception of treatment, and the second time point is after the inception of treatment. In some embodiments, the first time point is before the inception of treatment, and the second time point is after the completion of treatment. In some embodiments, the first time point is before a tumor resection surgery, and the second time point is after the tumor resection surgery. In some embodiments, the first time point is before a tumor resection surgery, and the second time point is about 5, 10, 15, 20, 25, or 30 days after the tumor resection surgery. In some embodiments, the first time point is before a tumor resection surgery, and the second time point is about 1, 2, 3, 4, 5, 6, 7, 8, 9, 10, 11 or 12 months after the tumor resection surgery. In some embodiments, the first time point is before a tumor resection surgery, and the second time point is about 1, 2, 3, 4, 5, 6, 7, 8, 9, or about 10 years after the tumor resection surgery.

In some embodiments, one or more changes of a mutational signature before and after administration of a treatment can be used to identify patient populations that respond better or worse to the treatment, according to a mutational signature classification. As such, tracking mutational signatures over time can be used to identify cases where therapy is ineffective, and to identify cases where a change in therapeutic intervention may be needed (e.g., administration of a different therapy may be needed).

In certain embodiments, a longitudinal mutation signature comprises a plurality of different time points, wherein a first time point is before the inception of treatment, and a plurality of additional time points are collected at specific time intervals following treatment, e.g., about 1, 2, 3, 4, 5, 6, 7, 8, 9, 10, 11 or 12 months following treatment. In some embodiments, a treatment comprises a tumor resection surgery with curative intent. In some embodiments, a treatment comprises administration of a therapeutic agent. In some embodiments, a therapeutic agent is an anti-cancer therapeutic agent.

In some embodiments, a longitudinal mutation signature comprises a plurality of different time points, wherein a first time point is before a tumor resection surgery with curative intent, and a plurality of additional time points are collected at specific time points following the tumor resection surgery, e.g., about 1, 2, 3, 4, 5, 6, 7, 8, 9, 10, 11 or 12 months or more following tumor resection surgery, such as about 1, 2, 3, 4, 5, 6, 7, 8, 9, or 10 years following tumor resection surgery. In some embodiments, a longitudinal mutation signature comprises a plurality of different time points, wherein a first time point is before administration of an anti-cancer therapeutic agent, and a plurality of additional time points are collected at specific time points following the administration of the anti-cancer therapeutic agent, e.g., about 1, 2, 3, 4, 5, 6, 7, 8, 9, 10, 11 or 12 months or more following administration of the anti-cancer therapeutic agent, such as about 1, 2, 3, 4, 5, 6, 7, 8, 9, or 10 years following administration of the anti-cancer therapeutic agent.

Aspects of the methods include collecting mutation signatures over time from asymptomatic patients to facilitate early detection of cancer and/or to predict risk levels associated with developing cancer. In some embodiments, a mutational signature is built up over multiple time points for an asymptomatic patient. The mutational signature can be used to estimate cancer or disease risk by, e.g., determining a status of certain genetic markers (e.g., BRCA germline status and somatic status) and/or the presence or absence of a cancer (e.g., a somatic mutation signature that is consistent with the presence or absence of a cancer) and/or a molecular classification of a cancer (e.g., a somatic signature coupled with a germline status determination).

Variables used in the creation of a mutation signature in accordance with embodiments of the invention include, but are not limited to, the total number of observed genetic variants, or alterations, the sequence context in which the variants occur, the prevalence of the mutation relative to other somatic mutations or to the germline genome, the type of genetic alteration, one or more fragmentation patterns of cfDNA fragments (e.g., a cfDNA fragment size distribution pattern, and/or the location of fragment start and end points), chromatin structure (e.g., open v. closed chromatin structure), methylation status, and inter-mutation distance (e.g., clustering of mutations.

Sequence context refers to the nucleotides surrounding the mutation. See, e.g., Sung et al., “Asymmetric Context-Dependent Mutation Patterns Revealed Through Mutation-Accumulation Experiments,” Mol. Biol. Evol., April 2015. By including the sequence context in which the variant occurs, mutation signatures with the same substitutions, but within different sequence context can be differentiated. For example, the genetic signature associated with UV damage evidences an increased number of C>T mutations with triplet context dependence (e.g., the substitution and the nucleotides 3′ and 5′ to the mutation). See Alexandrov et al. 2013. In some embodiments, the sequence context can include at least one, two, three, four, five, six, seven, eight, nine, ten, or more nucleotides on either or both of the positions 3′ and 5′ to the mutation. In some embodiments, a sequence context includes at least one nucleotide 3′ and at least one nucleotide 5′ to the mutation. In some embodiments, a mutation signature can take into account a strand on which a mutation occurs. For example, in some embodiments, a mutation can be more prevalent on a transcribed strand versus a non-transcribed strand. See Alexandrov at page 6.

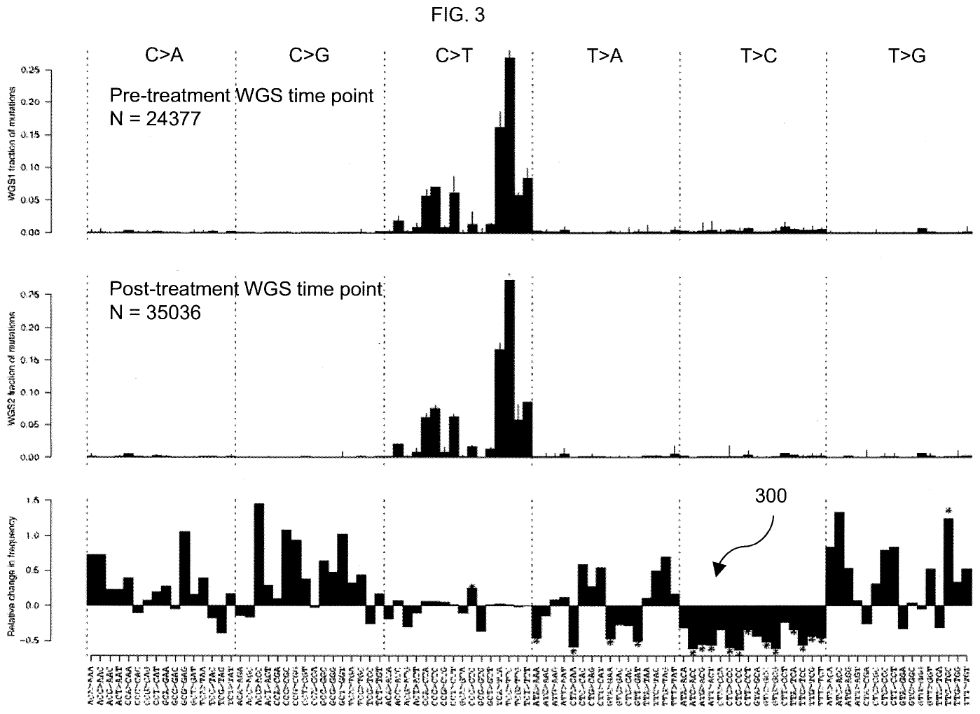

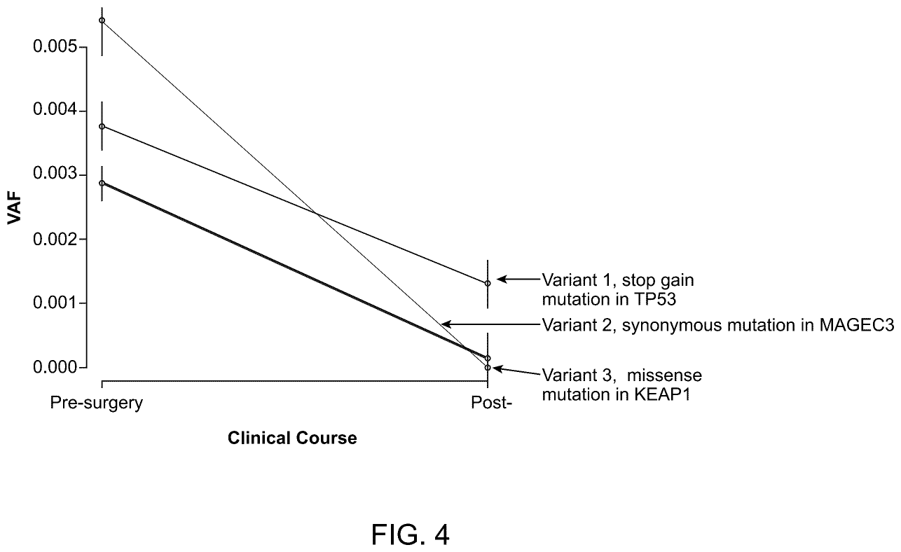

Longitudinal trajectories can be analyzed as the evolution of alterations stratified by sequence context. For example, FIG. 3 displays the dynamic melanoma mutation signature from cfDNA using whole genome sequencing (WGS) applied to a metastatic melanoma patient with a targetable somatic BRAF mutation V600R. Using WGS, mutations were identified and stratified by triplet context (Nₜᵢₘₑₚₒᵢₙₜ₁=24377, Nₜᵢₘₑₚₒᵢₙₜ₂=35036). Samples were taken and analyzed using WGS at a first timepoint prior to a course of treatment, as shown in the first panel, and then again at a second timepoint subsequent to a course of treatment, as shown in the second panel (95% Cl, bootstrapping). The observed profile was concordant with Type 2 melanoma reported by Alexandrov et al (2013) (cited herein) and is compatible with UV induced DNA damage. The profile exhibits abundant C>T mutations, as shown in the C>T column of FIG. 3. The relative change in frequency between time points was then calculated, as shown in the third panel, with the stars representing significant changes (p<0.05, FET). As can be seen, over the course of one year of treatment with vemurafenib (targeting BRAF) and ipilimumab (anti-CTLA4 checkpoint inhibitor), a systematic and consistent decrease in T>C mutations is observed in a patient evidencing with melanoma. The systemic and consistent change in the relative frequency of this mutation suggests potential differential response between sub-clones and or metastases in the patient. See, e.g., Venn et al., “Genome-wide cfDNA Sequencing of Melanoma Progression,” presented at the BioTrinity 2015 Conference in London on May 12, 2015, herein incorporated by reference in its entirety.

Furthermore, the prevalence of mutations is highly variable between and even within cancer types. For instance, certain childhood cancers are associated with the fewest mutations and cancers related to chronic exposures that cause mutations are associated with the highest number of mutations. See, e.g., Alexandrov at page 221. Furthermore, the prevalence of one mutation is variable with respect to other somatic mutations within a type of cancer. In accordance with the methods described herein, the prevalence of a mutation is measured by the variant allele frequency. The frequency, or prevalence, can then be compared to other mutations or to the germline genome (e.g., the ratio of circulating tumor DNA (ctDNA) to cell free DNA (cfDNA)).

Variant allele frequency is the relative frequency of an allele at a particular locus in a population. For example, to calculate an allele frequency across a population of individuals, one would calculate the fraction of all occurrences of an allele in i chromosomes in the population of N individuals with ploidy n, and the total number of chromosome copies across the population, represented by the following equation: Allele frequency=i/(nN).

For the frequency of a somatic mutation allele within an individual, the frequency is calculated as a quotient of the observed mutant allele copies (dividend) by the non-mutant allele copies in the individual. In some embodiments, the observed frequencies can be corrected for ploidy, noise-rates, and/or sub-clonal complexity.

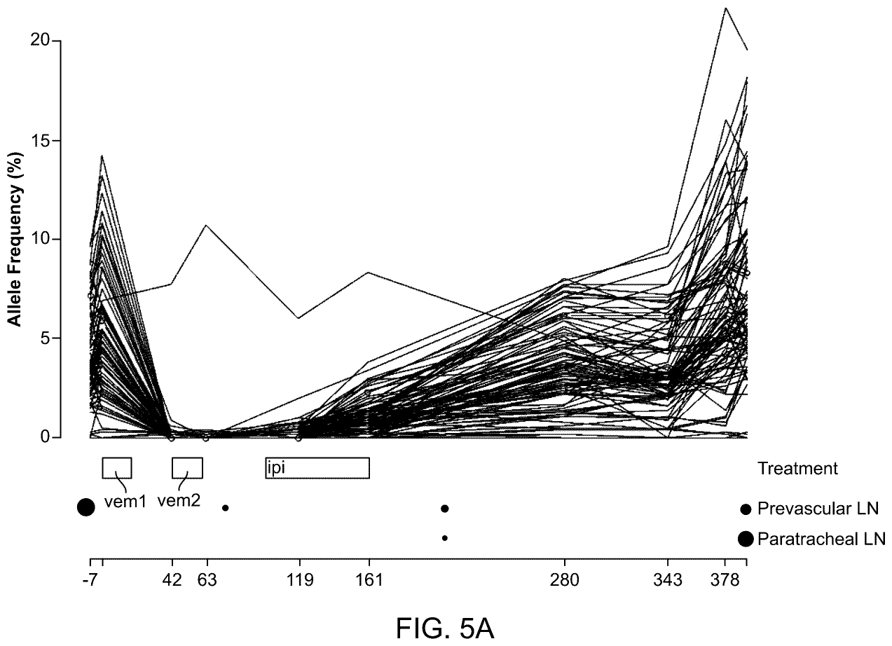

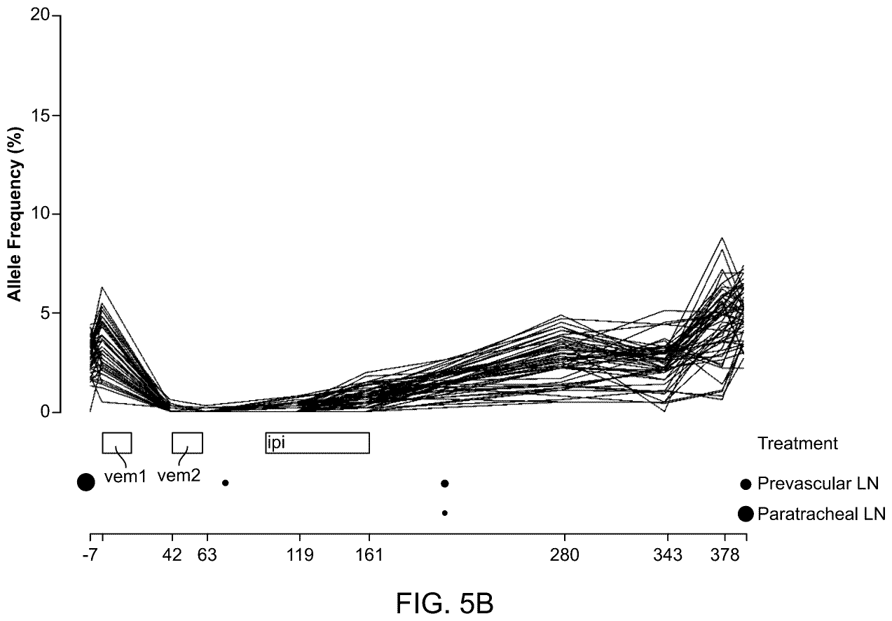

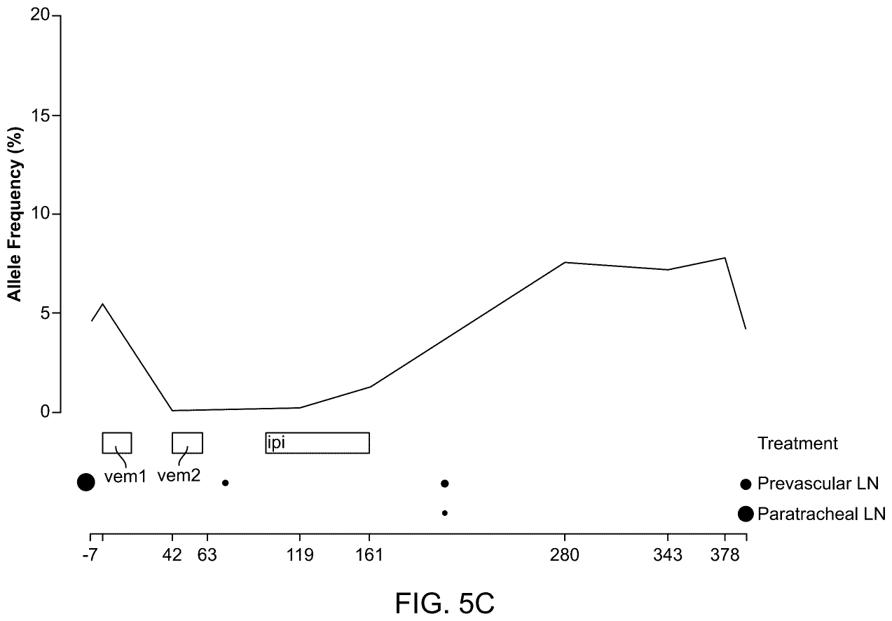

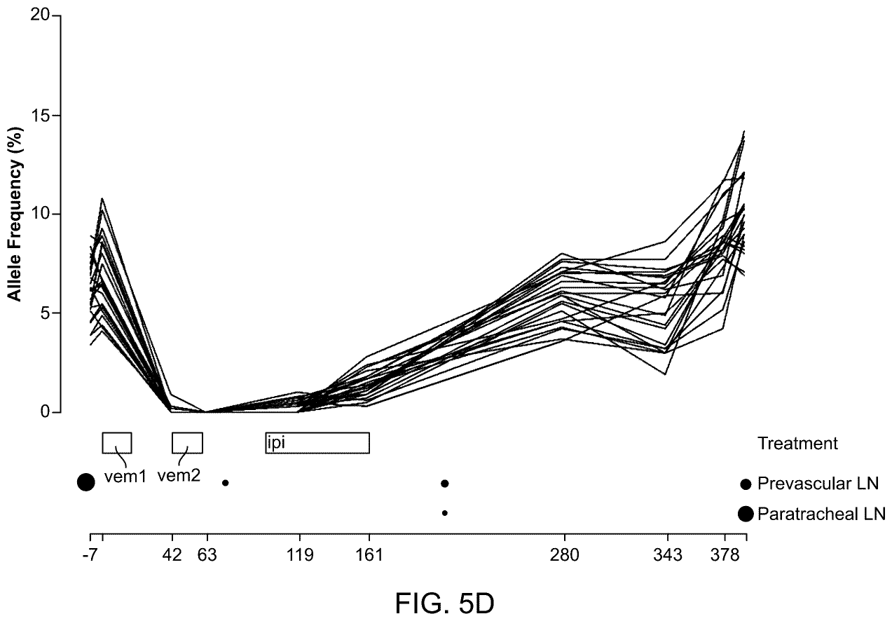

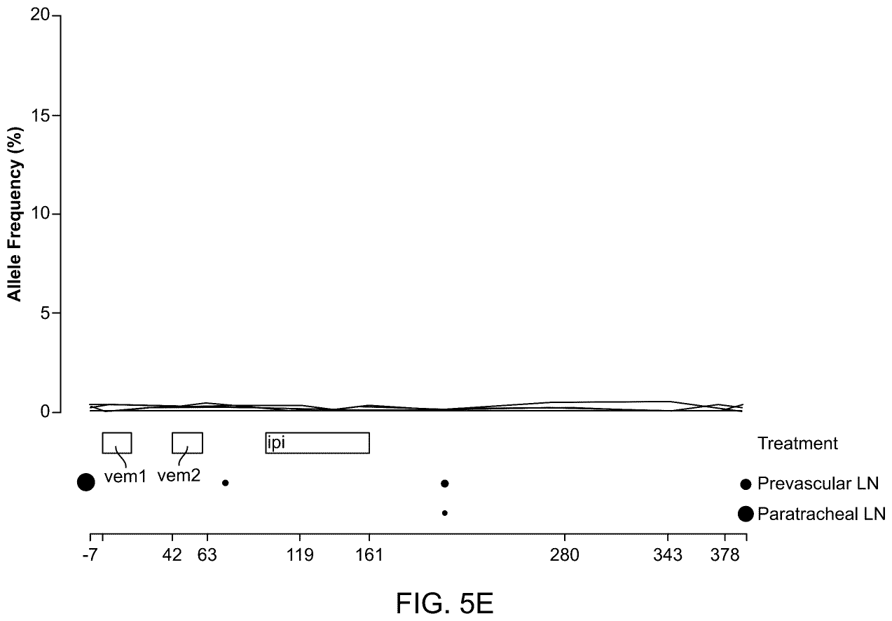

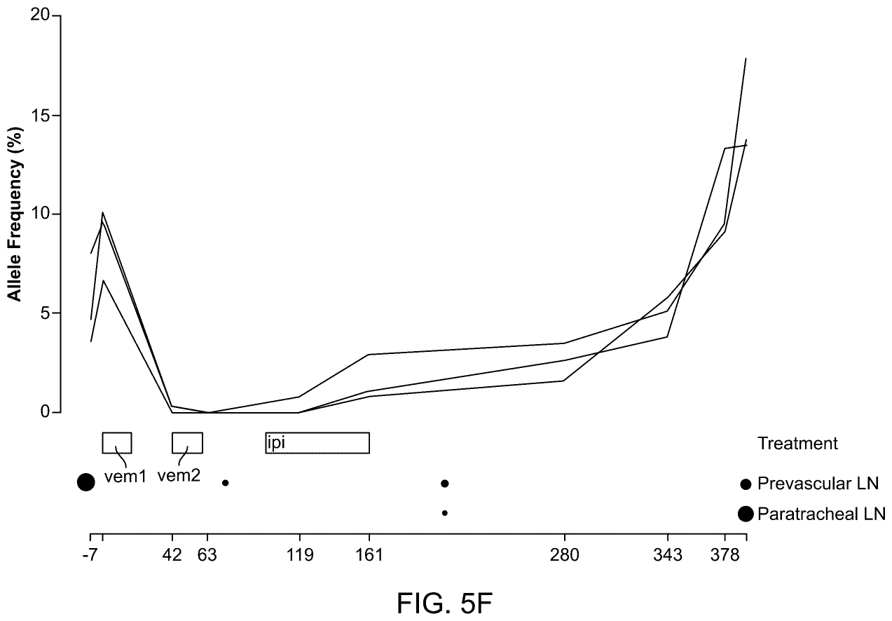

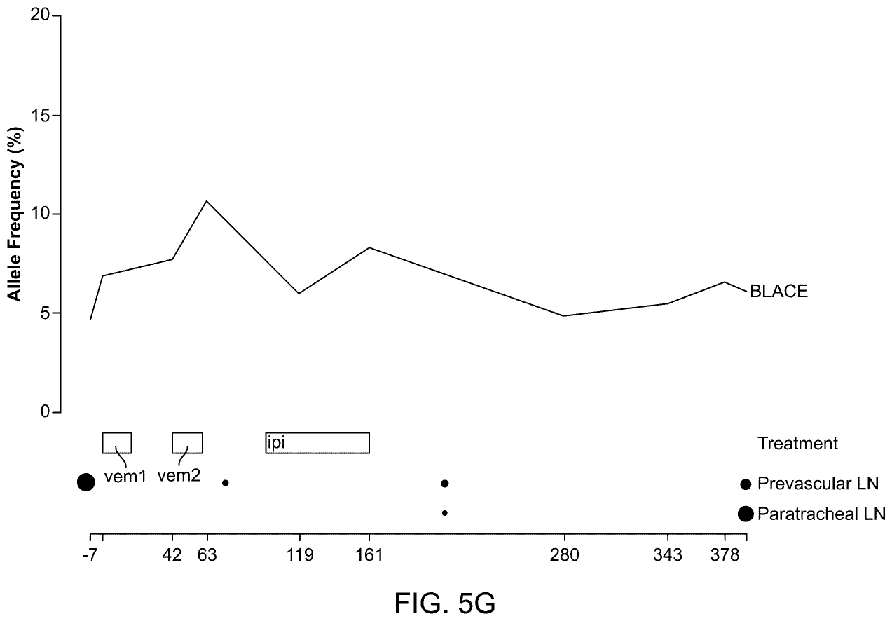

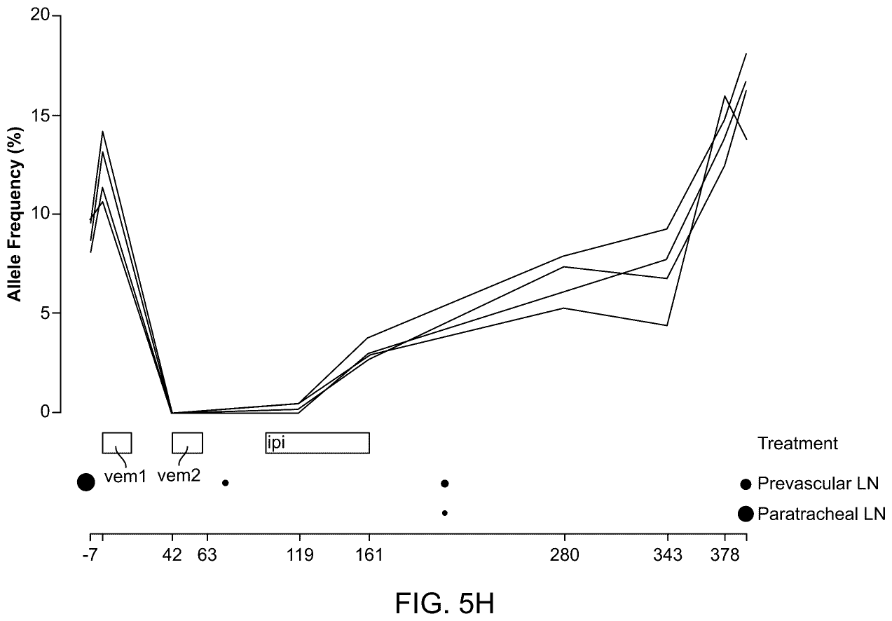

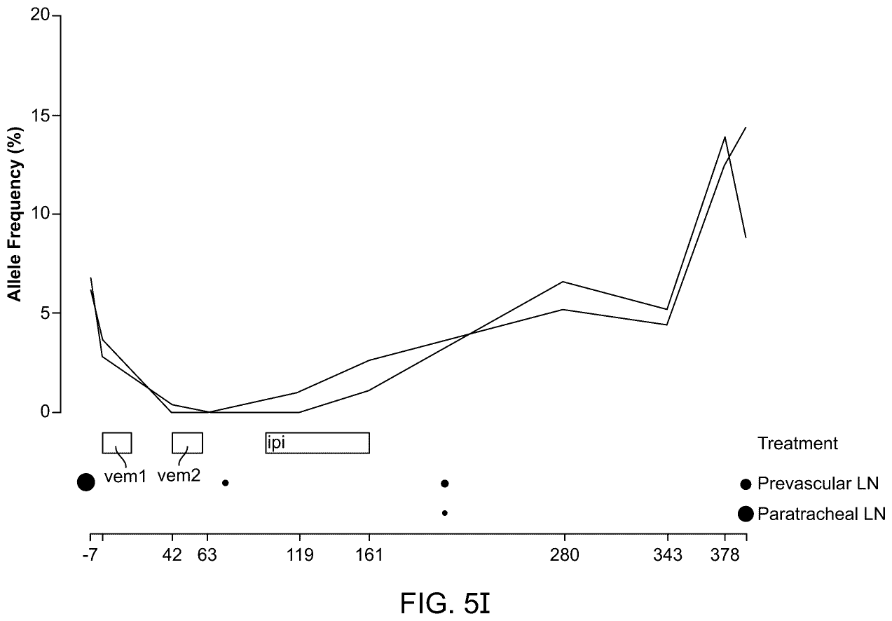

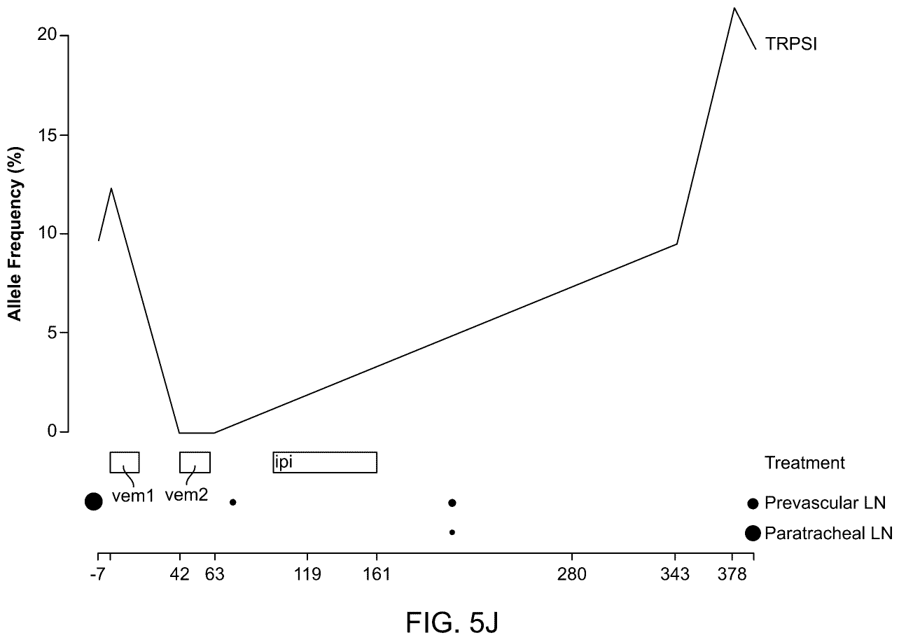

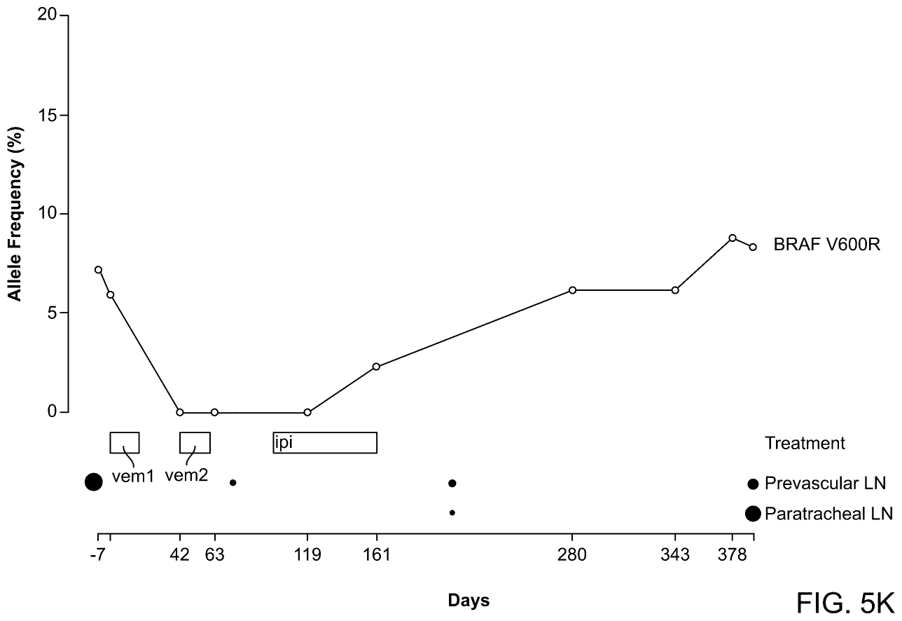

FIGS. 5A-K shows the allele frequency trajectories of 100 somatic mutations through the course of treatment. The variants were tracked using amplicon-based sequencing of cfDNA samples on PGM (Life Tech). Loci were assigned one of 8 clusters based on hierarchical clustering (Euclidean distance). Treatment cycles of vemurafenib (first two rectangles in the “Treatment” row, located above the x-axis) and ipilimumab (third rectangle in the “Treatment” row, located above the x-axis) are indicated in FIGS. 5A-K. Also shown are the tumor diameters for prevascular lymph node (“Prevascular LN” row, located above the x-axis) and paratracheal lymph node (“Paratracheal LN” row. located above the x-axis) obtained using CT imaging. Here, by tracking the allelic frequencies of the somatic mutations, it would have been possible to see early on that treatment with ipilimumab was ineffective. An increase in allelic frequencies was detectable 88 days before the third CT imaging scan. Variant allele frequency trajectory was highly correlated (86% Pearson correlation) with aggregated imaged lymph node diameter.

Furthermore, the type of genetic variation will also contribute to the classification of the tumor. Examples of genetic variations that can be used to classify tumors include, but are not limited to, telemoric sequence copy number status (explained in further detail below), single nucleotide polymorphism(s), chromosome instability, translocations, inversions, insertions, deletions, loss of heterozygosity, amplifications, kateagis (hyper mutation localized to small genomic regions; See Alexandrov), and microsatellite instability.

In addition to observed genetic variants, the classification can also include the determination of one or more gene product biomarkers, which provide different transformations of the underlying genomic information. A biomarker generally refers to a molecule that acts as an indicator of a biological state. In some embodiments, a gene product can be an RNA molecule or a protein.

Protein biomarkers in accordance with embodiments of the invention can include those proteins involved in oncogenesis, angiogenesis, development, differentiation, proliferation, apoptosis, hematopoiesis, immune and hormonal responses, cell signaling, nucleotide function, hydrolysis, cellular homing, cell cycle and structure, the acute phase response and hormonal control. See e.g., Polanski and Anderson, “A List of Candidate Cancer Biomarkers for Targeted Proteomics,” Biomark Insights, 1:1-48 (2007). Examples of cancer protein biomarkers approved by the FDA and encompassed by the present invention include, but are not limited to, CEA (carcinicenbryonic antigens); Her-2/neu; Bladder Tumor Antigen; Thyroglobulin; Alpha-fetoprotein; PSA; CA 125; CA 19.9; CA 15.3; leptin, prolactic, osteopontin and IGF-II; CD98, fascin, sPIgR, and 14-3-3 eta; Troponin I, and B-type natriuretic peptide. See Id; and Dawson et al., N Engl J Med 368:1199/1209 (March 2013).

Any assay known in the art can be used to analyze a gene product. In certain embodiments, an assay involves determining an amount of a gene product and comparing the determined amount to a reference. In one embodiment, a level of one or more protein biomarkers is obtained from a sample from the patient. The level obtained from the patient is then compared to a database of patient information of patients with known health statuses.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies can be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

The terms “differentially expressed gene” or “differential gene expression” refer to a gene whose expression is activated to a higher or lower level in a subject suffering from a disease, such as cancer, relative to its expression in a normal or control subject. These terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression can include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

In another embodiment, a MassARRAY-based gene expression profiling method is used to measure gene expression. For further details see, e.g. Ding and Cantor, Proc. Natl. Acad. Sci. USA 100:3059 3064 (2003). Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

Alternatively, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In some embodiments, Serial Analysis of Gene Expression (SAGE) is used to measure gene expression. For more details see, e.g. Velculescu et al., Science 270:484 487 (1995); and Velculescu et al., Cell 88:243 51 (1997, the contents of each of which are incorporated by reference herein in their entirety).

In some embodiments, Massively Parallel Signature Sequencing (MPSS) is used to measure gene expression. See e.g., Brenner et al., Nature Biotechnology 18:630 634 (2000).

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

In one aspect of the invention, the methods comprise the incorporation of patient information that can be used as covariates to assist in the classification. Non-limiting examples of patient information that can be incorporated include: age, gender, race, ethnicity, family disease history, weight, body mass index, height, prior and concurrent infections (e.g., HPV, HCV, EBV and HHV-6), environmental exposure(s) to potential toxins (e.g., asbestos exposure, ingestion of BPA from plastics, etc.), alcohol intake, smoking history, cholesterol level, drug use (illegal or legal), sleep patterns, diet, stress, and exercise history.

Patient information can be obtained by any means known in the art. In some embodiments, patient information can be obtained from a questionnaire completed by the patient. Information can also be obtained from the medical history of the patient, as well as the medical history of blood relatives and other family members. Medical history information can be obtained through analysis of electronic medical records, paper medical records, a series of questions about medical history included in the questionnaire, or a combination thereof. In some embodiments, patient information can be obtained by analyzing a sample collected from the patient, sexual partners of the patient, blood relatives of the patient, or a combination thereof. In some embodiments, a sample can include human tissue or bodily fluid.

In some embodiments, a health outcome is assessed for each patient. Health outcome can include one or more diagnoses of diseases or disorders and the stage or progression of the one or more diseases or disorders, or the outcome can be that the patient is otherwise healthy. Diagnoses are typically made by a medical practitioner/clinician and can be based on symptomatic, or clinical, observations, and/or laboratory results.

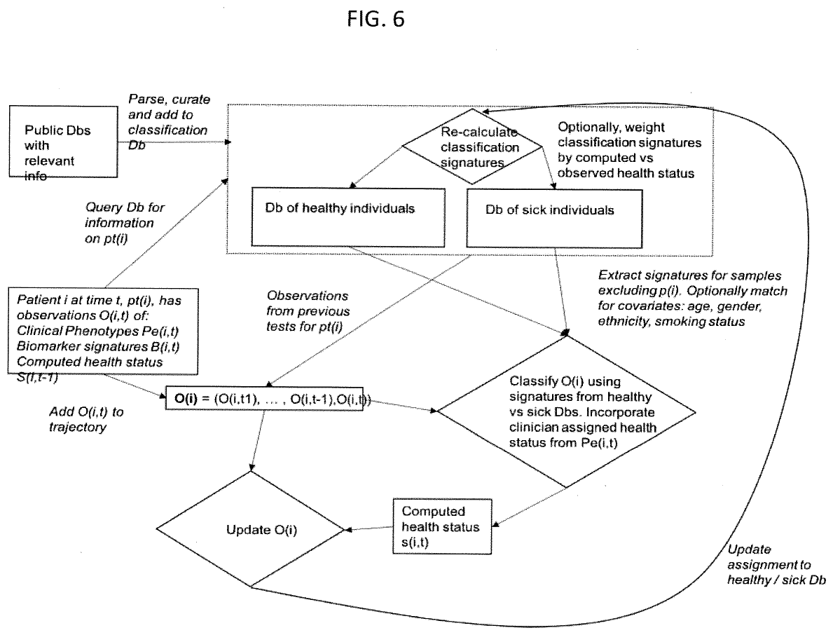

In accordance with some embodiments of the methods of the invention, and as shown in FIG. 6, patient data, which includes the observed genetic alterations, biomarker signatures, patient covariate information, and health outcomes, can be collected at various points in time. This data is used to generate a mutation signature (e.g. “classification signature”, as shown in FIG. 6) for the patient. The mutation signature is then compared to a database of healthy and sick individuals to compute the health status of that individual.

As the patient is followed through time, the computed health status can be refined according to the one or more databases or according to clinical information on the patient. This allows new disease signatures to be identified and refined through time. The classification database benefits from a network effect in that the discriminatory power of the one or more databases improves with each added patient and as patients are followed over time. As each additional patient's classification signature and health status is refined over time, that information can be entered into the one or more databases, such that the discriminatory power of the classification database(s) is improved.

As shown in FIG. 6, based on information obtained from public databases as well as information obtained from a database containing information observed directly from patients, classification signatures can be calculated. For example, the information obtained from public databases can initially be used to determine mutation signatures for both healthy and sick individuals. These signatures are to be stored in one database or can be stored in individual databases. When genetic data and other patient information is observed, and/or obtained from a patient (e.g., observed genetic variants, protein biomarker levels, clinician-determined health outcomes, and patient information, as discussed above), a mutation signature is created in accordance with embodiments of the methods of the invention. The information, data, and mutation signatures can be contained in a separate patient database or in multiple databases. The mutation signature of a patient can be pulled from the patient database(s) and compared to the database(s) of mutation signatures of healthy and sick individuals. The patient can then be assigned to either one of a category of healthy or sick individuals. Optionally, signatures can be weighted based on whether they were computed from public database information or observed directly from a patient. Over time, information from public databases and the patient information database(s) are used to inform the mutation signatures of healthy and sick individuals.

In some embodiments, information obtained directly from a patient is entered into a database(s) at each time point in which the information is obtained. These entries are used to create a longitudinal trajectory, or signature, such that the mutation signatures at each time point can be analyzed and compared to the mutation signatures in one or more database(s) of healthy and sick individuals over a period of time to determine a longitudinal mutation signature for a patient. Furthermore, the longitudinal signature for both the patient and the disease state can be refined over time as each observation(s) from a patient at a point in time is compared and added to the one or more databases of sick and healthy individuals.

In some embodiments, a computed health status for a patient can be determined using the mutation signature for the patient and based on comparison of that signature to a database(s) of mutation signatures for healthy and sick individuals. As noted above, the health status computation can incorporate the health outcome of the patient, as determined by a medical practitioner/clinician at various points in time. Both clinician-determined health outcomes and continued comparison to the database(s) of mutations signatures of healthy and sick individuals serve to refine the computed health status of a patient over time.

Refinement of mutation signatures and patient health statuses in accordance with the methods of the invention allows for the early detection of disease process, including intra-tumor and inter-tumor heterogeneity, and/or identification of minimal residual disease after treatment that otherwise would not be detectable using means currently employed in the art. Early detection is important in that it affords the opportunity for curative surgery and/or treatment, rather than detecting the disease at a more advanced stage, such as in metastases.

In some embodiments, the methods of the invention can be used to track the aging process in an individual. Mutations are observed in individuals as they age; these mutations not necessarily resulting in a cancerous development. By tracking a patient's genetic variations, phenotypic traits and environmental exposures, biomarker levels, and medical practitioner/clinician-determined health outcomes, longitudinal classification signatures (e.g. somatic burden scores) as they relate to aging can be created and refined.

As discussed above, tumors can be classified based, in part, on the type of genetic alteration, such as telomeric sequence copy number status. The telomeric sequence copy number status can also be used on its own to determine a diagnosis and/or proposed therapy for the patient, or the status can be combined with one or more of patient information, gene product biomarkers, and health outcomes, as discussed above with respect to the classification signature.

Telomeres are complex structures of DNA sequence and associated proteins that cap the ends of chromosomes and are critical for the maintenance of genome integrity. A telomeric DNA sequence is composed of repeated DNA motifs that vary between organisms. In humans, telomeres are typically 3-18 kilobases of (TTAGGG)n tandem repeats which are gradually eroded with cell doublings. Telomere sequence attrition leads to cell senescence of that cell.

Attrition is compensated by telomerase, a ribonucleotide-protein complex with reverse transcriptase activity that adds TTAGGG repeats on to the 3′ DNA end of chromosomes using its RNA component as a template. Telomerase is not usually expressed in somatic cells, but is present in stem cells and immortalized cells.

Reactivation of telomerase reverse transcriptase functionality is considered a fundamental step in oncogenesis (this enzyme is overexpressed in 85-90% of tumor cells). E.g., Akincilar et al., Cell Mole Life Sci, 2016. Other forms of telomere lengthening, such as alternative telomere lengthening, have also been observed in cancer patients. Consequently, there has been much interest in using telomere tandem repeat copy number as a biomarker of disease and aging.

There are several methods to detect dysregulation of telomeres. These include the use of polymerase chain reaction (PCR), restriction enzyme digestion, ligation of radiolabeled oligonucleotides, direct detection of telomerase activity, and immunohistochemistry techniques. Recently, methods have been described to estimate telomere length from WGS of genomic DNA. See, e.g., Zhihao Ding et al., Estimating telomere length from whole genome sequence data. Nucl. Acids Res. (14 May 2014) 42 (9): e75 first published online Mar. 7, 2014 doi:10.1093/nar/gkul81; Nersisyan L et al., (2015) Computel: Computation of Mean Telomere Length from Whole-Genome Next-Generation Sequencing Data. PLOS ONE 10(4): e0125201. doi: 10.1371/journal.pone.0125201; and Lars Feuerbach et al., TelomereHunter: telomere content estimation and characterization from whole genome sequencing data. 2016. bioRxiv 065532; doi: https://doi.org/10.1101/065532.

However, all the above-described approaches are limited in that they have only been applied in cross-sectional studies versus longitudinal studies. Much contradiction exists in the literature from cross-sectional cohort studies in different disease, disorder, and aging studies. Furthermore, the described approaches have only been applied to genomic DNA from peripheral blood mononuclear cells (PBMC). Such an approach only reflects telomere integrity in the leukocyte lineage.

In contrast, methods in accordance with embodiments of the present invention estimate telomere length from cell free nucleic acid (e.g., DNA, RNA) in a patient over time. The use of cell free nucleic acid to estimate telomere length reflects the consensus telomere integrity across all tissues in an individual, and not just a specific population, such as occurs with the use of PBMCs.

In accordance with one embodiment of the invention, telemore integrity from cell free DNA (cfDNA) is inferred by computing an integrity score from the sequencing of cfDNA. Any suitable method for sequencing cfDNA can be used in accordance with embodiments of the invention. For example, WGS can be used to sequence cfDNA. Such a method can be preferred due to the strong impact of GC content on PCR amplification bias and hybrid capture. Alternatively, a telomere integrity score can be computed by sequencing cfDNA that has been enriched for a specific telomere sequence or sequences, otherwise known as targeted sequencing. Telomeric sequencing can be enriched using PCR-amplification, hybrid capture, small molecules that bind to telomeric sequences, G-quadruplex signatures, or ChIP-seq with antibodies against telomere associated proteins.

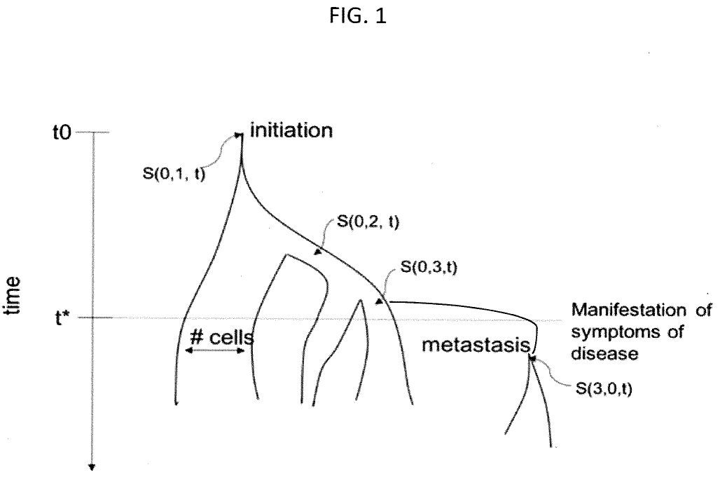

In some embodiments, a plurality of sequences can be aligned, using various alignment methods, such as those described in Zhihao Ding et al., Estimating telomere length from whole genome sequence data. Nucl. Acids Res. (14 May 2014) 42 (9): e75 first published online Mar. 7, 2014 doi:10.1093/nar/gkul81; and Nersisyan L et al., (2015) Computel: Computation of Mean Telomere Length from Whole-Genome Next-Generation Sequencing Data. PLOS ONE 10(4): e0125201. doi: 10.1371/journal.pone.0125201, both of which are incorporated herein by reference in their entirety. Both Ding and Nersisyan use whole-genome next generation sequencing (NGS) to generate short reads. In Ding, telomeric length is calculated by the TelSeq algorithm using the formula l=tₖsc, where l is mean telomere length, tₖ is the abundance of telomeric reads, s is the fraction of all reads with GC composition between 48% and 52%, and c is a constant for the genome length divided by the number of telomere ends. Ding at page 2. In Nersisyan, the short reads are used as input in the Computel algorithm, which are then mapped to a telomeric index that is built based on a user-defined telomeric repeat pattern and read length. The Computel algorithm then calculates the mean telomere length based on the ratio of telomeric and reference genome coverage, the number of chromosome, and the read length. Nersisyan at pages 2-4 and Nersisyan's FIG. 1.

It is also to be understood that other methods for identifying telomeric sequences known in the art can also be used in carrying out the subject methods. These include, but are not limited to, analysis of k-mer frequencies from de-novo assembly methods. See e.g., Li et al., Genome Res 20(2): 265-272 (2010); and Liu et al., published online at http://arxiv.org/abs/1308.2012 (2013), both of which are incorporated herein in their entirety. Using any of the above methods, sequence reads can be interrogated (directly or indirectly) for telomere specific tandem repeats.

In some embodiments, telomeric frequencies can be normalized for each individual. In one embodiment, the frequencies are normalized using the frequencies of control sequences that have the same proportion of individual nucleotides as the telomere-specific tandem repeat sequence. For example, the frequencies of a TTAGGG tandem repeat can be normalized using the frequencies of control sequences having the same A, C, G, and T proportions at the TTAGGG tandem repeat, but with a permuted sequence. In some embodiments, frequencies can be normalized by comparing a determined frequency distribution to a reference database of frequency distributions. In each case, the controls provide a reference frequency to which observed telomere frequencies can be compared and for which variation in the input amount of DNA can be accounted.

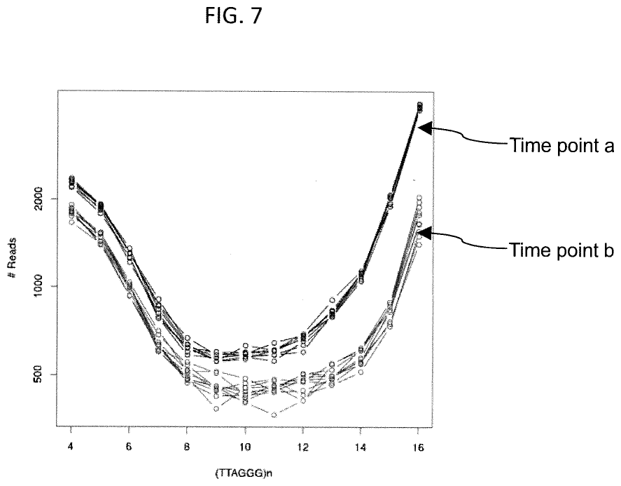

In some embodiments, an integrity score can be created once a telomere specific tandem repeat sequence is determined. The integrity score can contain a frequency distribution of telomere tandem repeat sequences as a function of repeat length. As with the classification signature discussed above, stratification can be done by sequence context, for example, by identifying sequences adjacent to telomeres on each chromosome arm. The topology of this distribution at any point in time, or its change between time points can be used as an identifying feature. FIG. 7 shows the empirical distribution of the number of whole genome sequencing reads from cfDNA containing repeated telomeric sequences from a melanoma cancer patient. Presented are two time points during treatment, identified by arrows. For each time point, the number of reads is calculated for each sequencing lane.

As with the classification signature discussed above, a longitudinal trajectory can also be constructed from the telomere integrity scores for each patient. This trajectory can then be compared to longitudinal trajectories contained in one or more databases of patients with known health statuses to determine a diagnosis and potential therapy. Furthermore, as discussed above with respect to the classification signature, patient information, gene product biomarkers, and health outcomes can also be integrated with the integrity score.

Information that can be obtained from the patient, to be used as, for example, covariates, can include but is not limited to age, gender, race, ethnicity, family disease history, weight, body mass index, height, prior and concurrent infections (e.g., HPV, HCV, EBV and HHV-6), environmental exposures to potential toxins (e.g. asbestos exposure, ingestion of BPA from plastics, etc.), alcohol intake, smoking history, cholesterol level, drug use (illegal or legal), sleep patterns, diet, stress, and exercise history. This information can then be compared to the one or more databases of patients with known health statuses.

Other covariates can include but are not limited to the input nucleotide mass and assay dynamic range. Alternatively or additionally the genetic background of the patient, such as the patient's TERT promoter mutation profile, can be included as a covariate.

Gene product biomarkers in accordance with methods of this invention can include protein expression levels. An example of a preferred protein is the telomerase protein. The level of the biomarker can be obtained from the patient according to any assay method known in the art, as described above. Once obtained, the level can be compared to the database of patients with known health statuses.

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

Functions described above can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the invention, a computer system 501 for implementing some or all of the described inventive methods can include one or more processors (e.g., a central processing unit (CPU) a graphics processing unit (GPU), or both), main memory and static memory, which communicate with each other via a bus.

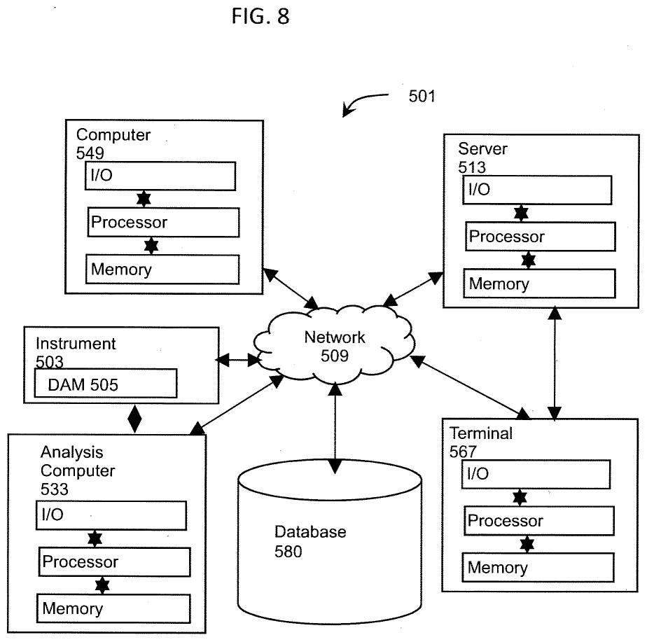

FIG. 8 provides a diagram of a system 501 according to embodiments of the invention. System 501 may include an analysis instrument 503 which may be, for example, a sequencing instrument. Instrument 503 includes a data acquisition module 505 to obtain results data such as sequence read data. Instrument 503 may optionally include or be operably coupled to its own, e.g., dedicated, analysis computer 533 (including an input/output mechanism, one or more processor, and memory). Additionally or alternatively, instrument 503 may be operably coupled to a server 513 or computer 549 (e.g., laptop, desktop, or tablet) via a network 509.

Computer 549 includes one or more processors and memory as well as an input/output mechanism. Where methods of the invention employ a client/server architecture, steps of methods of the invention may be performed using the server 513, which includes one or more of processors and memory, capable of obtaining data, instructions, etc., or providing results via an interface module or providing results as a file. The server 513 may be engaged over the network 509 by the computer 549 or the terminal 567, or the server 513 may be directly connected to the terminal 567, which can include one or more processors and memory, as well as an input/output mechanism.

In system 501, each computer preferably includes at least one processor coupled to a memory and at least one input/output (I/O) mechanism.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the invention can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database 580 included in the system. Any suitable database structure may be used including relational databases, object-oriented databases, and others. In some embodiments, reference data is stored in a relational database such as a “not-only SQL” (NoSQL) database. In certain embodiments, a graph database is included within systems of the invention. It is also to be understood that database 580 is not limited to one database; multiple databases can be included in the system. For example, database 580 can include two, three, four, five, six, seven, eight, nine, ten, fifteen, twenty, or more databases, including any integer of databases therein, in accordance with embodiments of the invention. For example, one database can contain public reference data, a second database can contain observed genetic variants, gene product biomarker levels, clinically assessed health outcomes and patient information from a patient, a third database can contain variant signatures of healthy individuals, and a fourth database can contain variant signatures of sick individuals. In another embodiment, the observed genetic variants, gene product biomarker levels, clinically assessed health outcomes and patient information can each be contained in a separate database. In yet another embodiment, the variant signatures of healthy and sick individuals are contained in one database. It is to be understood that any other configuration of databases with respect to the data contained therein is also contemplated by the methods described herein.

Cancer is a disease characterized by a complex lineage of genomic alterations, depicted schematically in FIG. 1. The processes of somatic mutation and somatic recombination generate genetic diversity within the tumor cell lineage. These alterations represent a discriminating and fundamental signature of the tumor—a molecular barcode.

Some alterations are causal, driving tumor progression, while other events have little functional consequence and are known as passenger mutations. The accumulation of alterations is observed as genetic heterogeneity within a tumor and/or between tumors in individual patients and between patients. Genetic diversity is an important contributor to treatment resistance, but also can generate neo-antigens that can be targets of host immune response.

Somatic genetic heterogeneity generates two challenges in tumor classification: tumors undergo rapid evolution through time; and, despite arising in the same tissue in two or more individuals, can be genetically distinct, with different prognoses and treatment response. FIG. 1 depicts the lineage of an initiating tumor cell. The ancestral cell arises at time t0, with genetically distinct sub-populations (subclones) arising during cell division and adding new branches to the tree. The relative population size of each subclone is represented by the width of each branch. Over time, three subclones are generated S(0,1), S(0,2), and S(0,3), each distinguished by its set of somatic alterations. If no reversion mutations occur and there is no recombination (recombination makes graphs), the mutations can be represented as a nested tree object (e.g. S(0,1) contained in S(0,3)). A metastasis S(3,0) is derived from rapidly expanding subclone S(3,0). The number of cells in S(0,2) decreases, contrastingly S(0,1) remains stable, and S(0,3) increases. Consequently, the frequency of a mutant allele will be a function of its relative frequency within and between subclones and healthy tissue. With the measurement of barcodes, the tumor can be detected before the manifestation of symptoms that only arise after multiple subclones have arisen.

Genetic tests, such as KRAS or BRAF mutation status, have demonstrated utility in therapy choice, for example, in cases such as the depicted case (PT0001, where BRAF mutational status was used to select vemurafenib therapy), informing the decision whether to use tyrosine kinase inhibitors. However, single locus tests are inadequate to capture the genetic heterogeneity in cancer, and therefore have limited utility in classification. Some studies have assessed heterogeneity using multi-region sequencing, while others have tracked pre-defined mutations through time.

Aspects of the present invention include a method to create a tumor classification signature from sampling part or all of the genetic variation in a patient through time. This longitudinal signature can be used to classify patient status against a database of signatures collected from known healthy and sick individuals. As each additional patient's signature and health status is refined over time, the next patient benefits from the improved discriminatory power of the classification database (FIG. 6). In FIG. 6, a flow chart depicts the transformation of observed genetic alterations, biomarker signatures, patient information and health outcome to generate a classification barcode for a hypothetical patient. Once determined, the classification barcode is then used to compute a health status according to a database of healthy and sick individuals. As the patient is followed through time, the health status can be refined according to the database, and/or according to clinical information from the patient. This allows new disease signatures to be identified and refined through time. The classification database benefits from a network effect in that the discriminatory power of the database improves with each added patient, and as patients are followed over time.

In a practical sense, the combinatorial complexity of the potential sequence of alterations and their sequence of occurrence, further compounded by their relative frequency, is infinite. Therefore, the construction of an abstract representation, the barcode trajectory, is required to make comparisons between previously observed cases and the patient under consideration.

Non-limiting examples of variables, or features, that can be used for classification include: the total number of observed variants; the sequence context in which the variants occur (e.g. UV damage has a signature of increased C>T mutations with triplet context dependence (Alexandrov et al., (2013) Signatures of mutational processes in human cancer. Nature, 500(7463), 415-421. http://doi.org/10.1038/nature12477); the prevalence (e.g., variant allele frequency) of the mutation relative to other somatic mutations, or to the germline genome (e.g., the ratio of circulating tumor DNA to cfDNA); the type of genetic alteration; telomeric sequence copy number status; chromosome instability; translocations; inversions; insertions; deletions; loss of heterogzygosity; amplifications; and microsatellite instability.

These genomic variables can be combined with protein biomarkers, e.g. CEA, or RNA signatures, which provide different transformations of the underling genomic information. From a database of observed signature trajectories, patient covariates (e.g., age, gender, smoking history), and health outcome (explanatory variable), an individual's tumor can be classified, its prognosis inferred, and potential therapeutic interventions can be inferred.

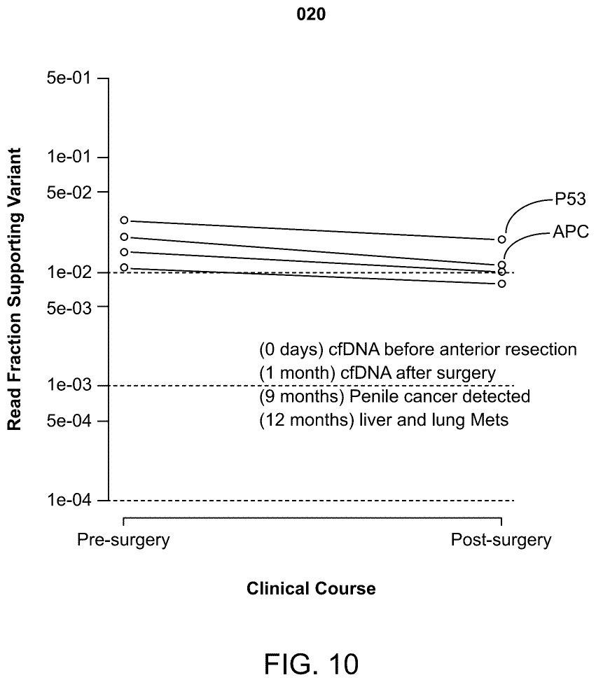

Cancer is characterized by a lineage of genetic aberrations manifesting as intra- and inter-tumor genetic heterogeneity. This diversity underpins treatment resistance, while also generating the reservoir of neo-epitope targets for cancer immunotherapies. Hence, constructing a measure of heterogeneity has important utility in patient care. Aspects of the present invention include methods for monitoring global treatment response by identifying and tracking heterogeneity signatures in a patient. Tracking multiple somatic alterations can help to guard against missing different intra- and/or inter-tumor responses in a patient, which improves the ability to detect minimal residual disease or treatment response (FIG. 10). For example, clustering can be accomplished using frequency-domain and/or time-domain methods. The clinical impact of tumor heterogeneity is that if only one trajectory is tracked, a clinician may conclude a partial response. However, in the depicted case, the patient presented with penile, liver and lung metastases at a follow-up that took place 6 months after surgery. Initial classification pT3 N0 (0/14) M0 L0 V0 R0 (wherein “pT3” indicates pathological staging with stage number 3; “NO” indicates the number of positive lymph nodes is zero (of 14 tested); “M0” indicates zero metastasis; “L0” indicates zero lymphatic vessel invasion; and “R0” indicates no residual tumor after resection). The depicted trajectories are compatible with, and demonstrate the presence of, at least one metastasis at the time of surgery. despite residual tumor classification of no residual tumor and neither positive lymph nodes nor metastasis.

Furthermore, trajectories can be analyzed as the evolution of alterations stratified by sequence context. FIG. 3 depicts a dynamic melanoma mutation signature obtained from whole genome sequencing (WGS) of cfDNA from a patient. FIG. 3 shows that over the course of 1 year of treatment with vemurafenib and ipilimumab, a systematic and consistent decrease in T>C mutations is observed, suggesting potential differential response between sub-clones and/or metastases in the patient. FIG. 3 depicts WGS-identified mutations stratified by triplet context (Nₜᵢₘₑₚₒᵢₙₜ₁=24377, Nₜᵢₘₑₚₒᵢₙₜ₂=35036). The observed profile is concordant with a Type 2 profile reported by Alexandrov et al. (2013), Signatures of mutational processes in human cancer. Nature, 500(7463), 415-421. http://doi.org/10.1038/nature12477, compatible with UV-induced DNA damage (abundant C>T, see upper right hand side insert). The first and second panels show mutational cfDNA WGS (bootstrapping, 95% CI). The third panel shows relative change in frequency between time points, stars represent significant changes (p<0.05, FET).

Aspects of the invention include methods for detecting cancer using telomere motif copy number from cfDNA. Telomeres are complex structures of DNA sequence and associated proteins that cap the ends of chromosomes and are critical for the maintenance of genome integrity. A telomeric DNA sequence is composed of repeated DNA motifs that vary between organisms. In humans, telomeres are typically 3-18 kilobases of (TTAGGG)n tandem repeats that are gradually eroded with cell doublings. Telomere sequence attrition leads to cell senescence of that cell.

Attrition is compensated by telomerase, a ribonucleotide-protein complex with reverse transcriptase activity that adds TTAGGG repeats on to the 3′ DNA end of chromosomes using its RNA component as a template. Telomerase is not usually expressed in somatic cells, but is present in stem cells and immortalized cells. Reactivation of telomerase reverse transcriptase functionality is considered a fundamental step in oncogenesis (this enzyme is overexpressed in 85-90% of tumor cells). Other forms of telomere lengthening, such as alternative telomere lengthening, have also been observed. Consequently, there has been much interest in using telomere tandem repeat copy number as a biomarker of aging and disease.

There are several methods for detecting dysregulation of telomeres, e.g., using PCR, restriction enzyme digestion, ligation of radiolabeled oligonucleotides, direct detection of telomerase activity, and immunohistochemistry techniques. Recently, methods have been described to estimate telomere length from WGS of genomic DNA (Ding et al., 2014; Nersisyan et al., 2015, both cited above).

However, all the above-described approaches have the limitation in that they have been described for a) cross-sectional studies and b) have been applied to genomic DNA from PBMCs, which is only a reflection of telomere integrity in the leukocyte lineage. There is much contradiction in the literature from cross-sectional cohort studies in different disease, disorder, and aging studies. Estimating telomere length from cfDNA reflects the consensus telomere integrity across all tissues in an individual. Aspects of the present invention include methods for constructing an inferred telomere integrity score from sequencing of cfDNA. In some embodiments, a telomere integrity score is computed from whole genome sequencing (WGS) of cfDNA. Due to the strong impact of GC content on PCR amplification bias and hybrid capture, WGS can provide more accurate results. In some embodiments, a telomere integrity score is computed from sequencing cfDNA that has been enriched for a telomeric sequence (i.e., targeted sequencing). Telomeric sequences can be enriched using PCR-amplification, hybrid capture using selectable oligonucleotides (e.g., biotinylated), using small molecules that bind to a telomeric sequence and/or G-quadruplex structures, or using ChIP-seq with antibodies against telomere associated proteins.

In some embodiments, a telomeric sequence can be identified using alignment-based methods, as described by Ding et al. (2014) or Nersisyan et al. (2015), both cited above, or through the analysis of k-mer frequencies from de-novo assembly methods known to the art. In both instances, sequencing reads are interrogated (either directly or indirectly) for telomere-specific tandem repeats. Telomere frequencies can be normalized per individual using the frequency of control sequences that have the same A, C, G, and T proportions at the TTAGGG tandem repeat, but with permuted sequence, or by targeting a unique homozygous locus in the genome. These controls provide a reference frequency against which telomere frequencies can be evaluated, and to account for variation in the input amount of DNA.

Aspects of the invention include methods of constructing a longitudinal trajectory of the telomere integrity score for each patient. The trajectory of the individual can then be classified against a reference database of other individuals who have known health outcomes. The integrity score can contain the frequency distribution of telomere tandem repeats as a function of repeat length, potentially stratified by an identifying sequence adjacent to telomeres on each chromosome arm. The topology of this distribution at any point in time, or its change between time points, can be used as an identifying feature.

In one preferred embodiment, the subject methods involve isolating cfDNA from a blood plasma sample of a patient, and performing sequencing of the cfDNA using Illumina sequencing.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

The following examples are offered for illustrative purposes only, and are not intended to limit the scope of the present invention in any way. While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. Various examples of changes, substitutions, and alterations are ascertainable by one skilled in the art and could be made without departing from the spirit and scope disclosed herein.

All references cited throughout the specification are expressly incorporated by reference herein.

### Materials and Methods

The following materials and methods were used in the examples described below.

Design: cfDNA from blood samples from 10 colorectal cancer patients was sequenced before and after surgery (listed in Table 1), one renal cancer patient (#004) and one breast cancer patient (#009). Patient clinical information is reported in Table 1.

Sequencing Method: Sequencing libraries were generated from 70 ng of cfDNA that was isolated from pre- and post-surgery blood plasma samples per protocol SENTRYSEQ version 1. The protocol is comprised of seven stages: plasma separation from blood, extraction of cfDNA from plasma, sequencing library preparation, quality control checks, PCR-amplification, target enrichment, and sequencing. Stages are described in order.

Blood was collected in 10 mL EDTA tubes and the samples processed to separate plasma within 2 hours of blood draw in order to minimize contamination from genomic DNA. Plasma was extracted using centrifugation: first, blood was centrifuged at 3000 rpm for 10 minutes at room temperature minus brake; second, plasma was transferred to 1.5 mL tubes in 1 mL aliquots and run second spin at 7000 rpm for 10 minutes at room temperature. The supernatant was then transferred to new 1.5 mL tubes, which can be stored at −80 C. Cell free DNA was extracted using Qiagen QIAmp Circulating Nucleic Acid kit, with modifications to the elution protocol to maximize cfDNA yield from input material. cfDNA was extracted using the Qiagen QIAmp circulating nucleic acid kit following the manufacturer's instructions (maximum amount of plasma allowed per column is 5 mL). If cfDNA was being extracted from plasma where the blood was collected in Streck tubes, the reaction time with proteinase K was doubled from 30 min to 60 min. If there was enough material, the maximum allowed volume of 5 mL was filled. The protocol was then modified to a two-step elution to maximize cfDNA yield: first, DNA is eluted using 30 μl of buffer AVE for each column (official protocol 20-150 uL). The amount of buffer used was minimized in the elution while ensuring complete coverage of the membrane. This limits dilution to maximize cfDNA concentration, avoiding the requirement for downstream desiccation of samples, which can cause double stranded DNA melting and or material loss. Second, 30 uL of buffer AVE was eluted for each column. The second elution increases DNA yield as shown in Table 1. Additional elutions must be balanced by decreasing the final DNA concentration in the elution. Elutions are then combined. Quantify extracted DNA in triplicate using the Qubit DNA HS kit. Modify Illumina's TruSeq Nano kit (Part #15041110 Rev. B) for WGS. Vendor supplied Illumina's TruSeq Nano kit was used to prepare libraries. This kit is designed for whole genome sequencing, but the reagent stoichiometry and incubation times were modified to increase the number of molecules with correct sequencing adapter ligation through the process (library conversion efficiency). No fragmentation of sampled DNA (e.g., sonication) was performed, since cfDNA is already fragmented (average length of cfDNA population about 167 bases and the distribution of fragment sizes varies from individual to individual). No SPRI bead cleanup steps before End Repair to minimize loss of cfDNA. This eliminates the risk of ethanol carry over into PCR; ethanol is a well-known inhibitor of PCR and it is challenging to remove all Ethanol droplets before SPRI beads start to crack. Also results in less hands on time. Adjusted Illumina reagent volumes based on the estimated number of DNA fragments in the sample by factor A to account for the different number of cfDNA fragments N_f relative to the fragments from sonicated genomic DNA N_g specified in TruSeq Nano protocol. Adjustment applied to reagents using in End Repair, 3′ End Adenylation, and Adapter Ligation steps. The number N_i of molecules in population i is calculated by dividing the mass of the population m_i by the product of the average molecular weight of one dideoxyribonucleotide (w=6.5E+11 ng/mole) and the average number of bases in each molecule L_i, then multiplying this value by Avogadro's constant N_i=m_i/(w×L_i)×N_A. The adjustment factor A is the quotient of NJ divided by Ng, A=m_f/m_g×L_g/L_f. The Illumina TruSeq Nano kit protocol which specifies m_g=100 ng of input DNA, and specified sonication to fragment length of L_g=350 bases. To maximize the number of cfDNA fragments that have at least two Y-shaped Illumina sequencing adapters ligated for paired-end sequencing, the adapter ligation reaction time was increased to 16 hours and the kinetic energy of the molecules in solution was decreased using a lower incubation temperature of 16 C.

Adapter ligation resulted in ‘stacking’, after PCR amplification the multiple stacked adapters were converted to single adapter copies on each end of the molecules through steric hindrance. Samples were cleaned up using SPRI sample purification beads at a ratio of 1:1.6 and then 1:1 of sample:beads, which was optimized to remove free adapters. At the final step of the library preparation, the mixture was eluted into a recommended volume of 27.5 μl. This concludes the library preparation step.

Next, the fragment size distribution of the DNA population was recorded using a Bioanalyzer (or equivalent) instrument. Readouts from this machine before and after PCR amplification of the sequencing library show that stacked adapters occur and are effectively resolved through PCR, resulting in a higher yield of molecules that are compatible with paired-end sequencing, which are referred to as “sequencable” molecules. For fragment size determination, 1 μl of cfDNA was input to identify average fragment length pre- and post-library preparation. The distribution of cfDNA molecule lengths prior to sequencing library preparation can be approximated as sampling from a Normal distribution, X_pre˜N(μ_pre,α\hat{ }2), with mean length μ_0 of about 150-180 bases, and sample variance α\hat{ }2. The distribution of molecule lengths post library preparation, X_post, can be approximated as a superposition of Normal distributions shifted by the number of ligated sequencing adapters, each sequencing adapter has fixed length A, which is usually 60 bases for Illumina platforms (P5 and P7 adapters). Molecules that can be sequenced (sequencable) have at least 1 adapter ligated to each end of the cfDNA fragment, thus having a mean of μ_0,+kA, where k≥2. As described in the Adapter Ligation section, if the library is PCR amplified, sequencable molecules are generated if the number of ligated adapters, k, is at least 2: X_post˜Σ_(k=0)\hat{ } 4 Y_k×N((μ_pre+kA),α\hat{ }2), k∈N_0, where Y_k is the weight of the contribution of molecules with k adapters ligated. After PCR amplification using P5 and P7 PCR primers the population is dominated by the population μ_pre+2A.

Next, the library is quantified. The mass of the library is quantified using a Kapa Library Quantification Kit (Kapa Biosystems). Quantification is important in determining library yield through the library preparation process and for calculating the reaction volumes for subsequent steps in the protocol. Kapa HiFi Hotstart amplification (Kapa Biosystems, KR0370-v5.13) was used for amplification. High fidelity PCR enzymes with robust performance across GC content are used. High Fidelity enzymes such as Kapa HiFi Hotstart have 100× lower error rates that Taq. The level of duplicate reads impacts the total amount of required sequencing. A simulation engine was used to assess the optimal over-amplification factor to detect variants at specified frequencies, jointly incorporating losses during library prep, induced errors, and the calling algorithm dependencies. The ratio of reads to underlying original molecules in an ensemble was referred to as the Over-amplification Factor. To calculate the number of samples that can be analysed on one sequencing run, the following formula is applied: (samples/run)=[(reads/run)÷((#genome equivalents/sample)×(panel size)×(overamplification factor))/average library molecule length]. This ensures efficient utilization of each sequencing run, while ensuring that there are enough reads for ensembles to be represented in the sequencing. The PCR amplification is as follows. The number of PCR cycles required to achieve desired redundancy is calculated using a model fit to previous PCR runs. First calculate PCR efficiency by fitting exponential model to known input amount of cfDNA. Then using the estimated parameters calculate total number of amplifications required to achieve desired overamplification (average number of PCR duplicates per original input molecule). 20 ul of each sample was used in the subsequent 8 cycle PCR: 25 uL of KAPA HiFi Mastermix 25 uL, 2.5 uL of 10 uM Forward primer, 2.5 uL of 10 uM Reverse primer, 20 uL of Template DNA.

Samples were cleaned up using Sample purification beads at a ratio of 1:1.6 and eluted into a volume of 22 uL. 1 ul was run on a Bioanalyzer and 3 uL was used to quantify library concentration by qPCR in triplicate.

Next, Pull-down by hybridization capture was performed. Mutation hotspots were identified across cancer types and combined with models of determinants of hybrid capture performance using IDT's protocol (DNA Probe Hybridization and Target Capture, version 2.0) to design a custom hybrid capture panel. To further optimize performance of the hybrid capture panel, the stoichiometric ratio of hybrid capture probes to the input sequencing library was optimized. The input amount of probe was decreased under the hypothesis that limiting the input probe amount would decrease off target pulldown thereby increasing specificity. The capture was observed to be fairly robust to hybrid capture probe concentration. The incubation time for hybrid capture was from 4 to 16 hours at 60 C incubation temperature. The combined bioinformatics optimization and reaction condition optimization increased yield to 47% with and on target rate of 80% and uniformity of 1.6 (estimated as maximum fold change in sequencing depth of reads for 95% of the sequenced population). Since each molecule was represented by approximately 8 copies, on average 4 copies were retained across the panel, given the consistent coverage uniformity.

The protocol for the hybrid capture is specified below: 500 ng of prepared sequencing library, 5 ug Cot-1 DNA and 1 uL of each Universal oligo were dried down in the speedvac. It is essential that the library is not dried out as this melts DNA. Resuspend contents of tube in 2×7.5 ul hybridization buffer, 3 uL hybridization component and 2.5 uL nuclease free water. Resuspended material was incubated in a thermocycler at 95° C. for 10 minutes. Added 3 pmol Lockdown Xgen probe (IDT, CA) to the solution. Incubated hybridization reaction at 60° C. for 16 hr. Followed IDT protocol for binding target to streptavidin beads and wash steps. For each sample, an aliquot was taken and quantified by qPCR followed by 12 cycles of PCR on 20 ul of library (same conditions as described above). Carried out clean-up with Agencourt Ampure XP beads. Final library is eluted into 22 uL IDTE. 1 uL then run on a Bioanalyzer to determine size distribution and quantified by qPCR using P5, P7 primers in triplicate. Sequencing. Samples were diluted to 2 nM initially and then a final concentration of 19 pM in 600 uL was loaded onto the HiSeq. This results in optimal cluster generation on HiSeq2500. However, if desired cluster generation is 850-1000 K/mm2 on a rapid run is not obtained the loading concentration may have to be varied.

TABLE 1
cfDNA yield from second elution from the Qiagen QIAmp Circulating
Nucleic Acid kit. cfDNA samples from six melanoma patients.
Elution volumes were both 30 uL of AVE.

| | Plasma volume | Elution 1 | Elution 2 |
|---|---|---|---|
| Sample ID | (mL) | (ng) | (ng) |
| Plasma 009 | 3 | 12.63 | 5.22 |
| Plasma 010 | 3 | 11.76 | 6.12 |
| Plasma 045 | 3 | 21 | 4.14 |
| Plasma 020 | 3 | 20.94 | 5.7 |
| Plasma 062 | 3 | 17.1 | 5.88 |
| Plasma 063 | 3 | 18.9 | 6.6 |

This protocol applies PCR amplification to create multiple copies of original cfDNA molecules, followed by a hybridization capture step that utilizes pulldown capture enrichment of targeted genomic regions. Samples were paired-end sequenced on a HiSeq2500 instrument (Illumina, CA) in HT mode.

Sequencing Data: Pre- and post-surgery samples are identified by numeric sample IDs with “pre” or “post” suffix. FASTQ files were downloaded from BaseSpace. Reads were aligned to a human reference genome, the “1000 Genomes Human Reference Genome”, (build 37) using sample BWA (version 0.7.8). Alignment BAMs were sorted, merged and indexed using Samtools (version 1.2) and Picard (version 1.111). Sequencing summary statistics were generated using Picard (version 1.111). Candidate somatic variants were called from alignments in cfDNA. A list of nine samples and their characteristics are provided in Table 2, below.

TABLE 2
List of samples.

| | Post-surgery 16 Dec. 2015 | |
|---|---|---|
| Patient | 126 base PE reads | |
| ID No. | Reads | GB |
| 041 | 273,997,742 | 17.71 |
| 210 | 401,354,832 | 26.20 |
| 225 | 340,101,890 | 22.56 |
| 030 | 348,268,676 | 22.44 |
| 034 | 304,033,890 | 19.71 |
| 015 | 341,846,560 | 21.89 |
| 088 | 296,138,376 | 19.06 |
| 187 | 371,512,946 | 23.95 |
| 196 | 405,343,732 | 26.51 |

### EXAMPLES

### Example 1: Detection of Disease Recurrence and Presence of Metastases Using cfDNA Somatic Variant Frequency Trajectories in Patient ID No. 034

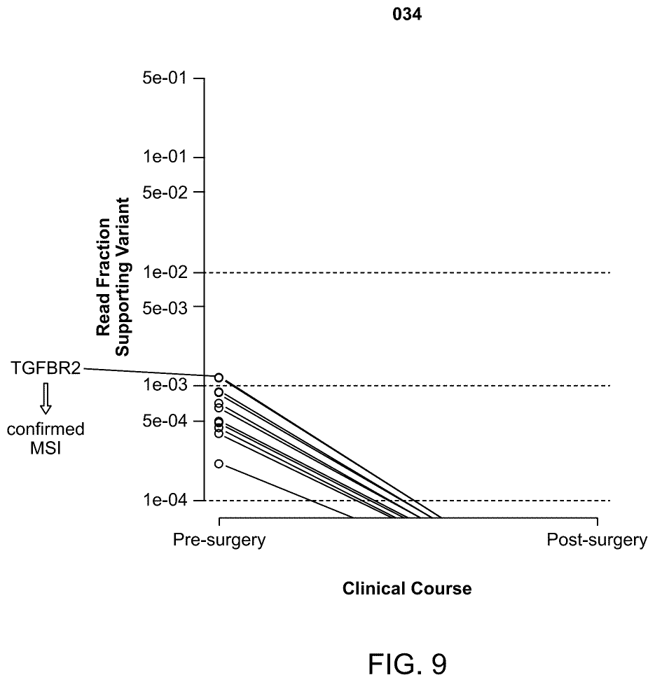

Colorectal cancer (CRC) patient ID No. 034 underwent surgery with curative intent. Pre- and post-surgery blood samples were collected, and cfDNA therein was sequenced as described above. Pre-surgery sequencing data revealed thirteen detected somatic variants. The allele frequency of all thirteen detected somatic variants decreased to non-detectable levels in the post-surgery sample, indicating complete resection of the tumor. The results are showing in FIG. 9. FIG. 9 shows the change in the fraction of reads containing a somatic mutation pre- and post-surgery. Each circle and connecting line represents an individual somatic mutation. Genes with mutations that have computationally inferred functional impact are identified. One identified mutation was in TGFBR2, with high functional impact. Researchers have investigated whether inactivation of TGF-beta receptors is a mechanism by which human colon cancer cells lose responsiveness to TGF-beta. See, e.g., Markowitz et al. (1995), Inactivation of the type a TGF-ß receptor in colon cancer cells with microsatellite instability, Science 268 (1995): 1336-1338. Results have demonstrated that the TGFBR2 gene was inactivated in a subset of colon cancer cell lines (referred to as RER+, for “replication errors positive”), exhibiting microsatellite instability, but not in RER(−) cells.

### Example 2: Detection of Disease Recurrence and Presence of Metastases Using cfDNA Somatic Variant Frequency Trajectories in Patient ID No. 020

Colorectal cancer (CRC) patient ID No. 020 underwent surgery with curative intent. Pre- and post-surgery blood samples were collected, and cfDNA therein was sequenced as described above. Pre-surgery sequencing data revealed a plurality of detected somatic variants. However, after surgery, the allele frequencies of the detected somatic variants did not decrease to non-detectable levels (in contrast to patient 034, Example 1). Results are shown in FIG. 10. FIG. 10 shows the change in the number of reads containing a somatic mutation pre- and post-surgery. Each circle and connecting line represents an individual somatic mutation. Genes with mutations that have computationally inferred functional impact are identified.

Nine months after surgery, a secondary tumor was detected. After 12 months, liver and lung metastases were detected. The trajectories on the graph in FIG. 10 indicate that the primary tumor and the metastases were clonally uniform, meaning they contained the same allele frequencies of the same somatic variants. These results demonstrate the value of using cfDNA sequencing analysis to determine a trajectory of a given somatic mutation when sampling multiple tumors within a patient. For this patient, the trajectories indicated that residual disease was still present.

### Example 3: Detection of Disease Recurrence and Presence of Metastases Using cfDNA Somatic Variant Frequency Trajectories in Patient ID No. 187

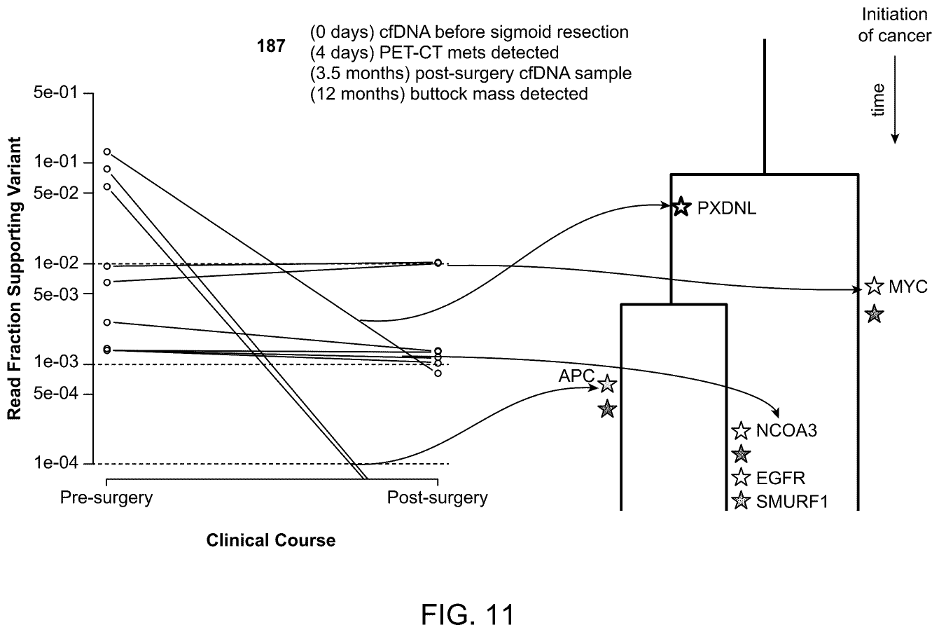

Colorectal cancer (CRC) patient ID No. 187 underwent surgery with curative intent. Pre- and post-surgery blood samples were collected, and cfDNA therein was sequenced as described above. Patient 187 displayed a diverse trajectory response for 9 somatic variants pre- and post-surgery that reflects a history of metastatic development in missed metastatic colorectal cancer. Results are shown in FIG. 11. FIG. 11 shows the change in the number of reads containing a somatic mutation pre- and post-surgery. Each circle and connecting line represents an individual somatic mutation. Genes with mutations that have computationally inferred functional impact are identified.

Clinical history at a plurality of different time points is provided in the top panel of FIG. 11. The treating surgeon did not know of the metastases prior to surgery with curative intent. Three allele frequency clusters in the pre-surgery cfDNA sample indicate the presence of three distinct cancer cell populations. A differential change in allele frequency after surgery confirmed that the three clusters arose from three different tumor populations. The tree in the right-hand panel of FIG. 11 represents a potential underlying lineage of the cancer cells within the patient, with time progressing from top to bottom, and the left most lineage in the tree representing a tumor that was surgically resected. The ancestral mutation in PXDNL is identified as the frequency approaches the frequency of mutations in the middle lineage. The far right lineage does not share any tumor mutations with the resected tumor, and therefore is unchanged after surgery, indicating that residual disease is still present.

### Example 4: Microsatellite Instability (MSI) in cfDNA Samples

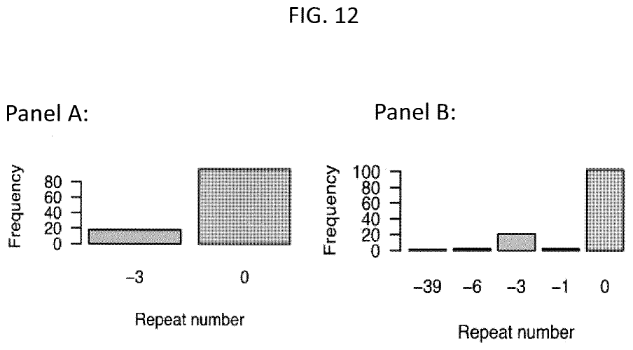

Analysis of cfDNA from a patient with microsatellite instability (MSI) using the lobSTR program (Gymrek et al., (2012), lobSTR: a short tandem repeat profiler for personal genomes, Genome research 22.6 (2012): 1154-1162) showed evidence of more than two alleles at the locus chr20:3,345,703 (FIG. 12, Panel B), demonstrating that MSI can be directly observed in cfDNA. In contrast, a sample from a cancer patient with no MSI (negative control) shows no evidence for MSI in cfDNA. Differences in frequency in FIG. 12, Panel A can be driven by differential hybridization capture efficiency of non-reference repeat elements. Microsatellite instability is identified as short tandem repeats (STRs) that are present in more than 2 alleles in cfDNA.

FIG. 12, Panels A & B show the distribution of (TGC)n repeat allele frequencies at chr20:3,3345,703 (Human Reference B37) in a patient that has no evidence for MSI through clinical testing (FIG. 12, Panel A), and a patient that has confirmed MSI through clinical testing (FIG. 12, Panel B). The Y-axis represents relative change in repeat number: a repeat number of zero represents the same repeat number as observed in the human genome reference, whereas values less than zero represent a decrease in the number of copies (deletions), and values greater than zero represent a relative increase in the number of repeat copies at that locus.

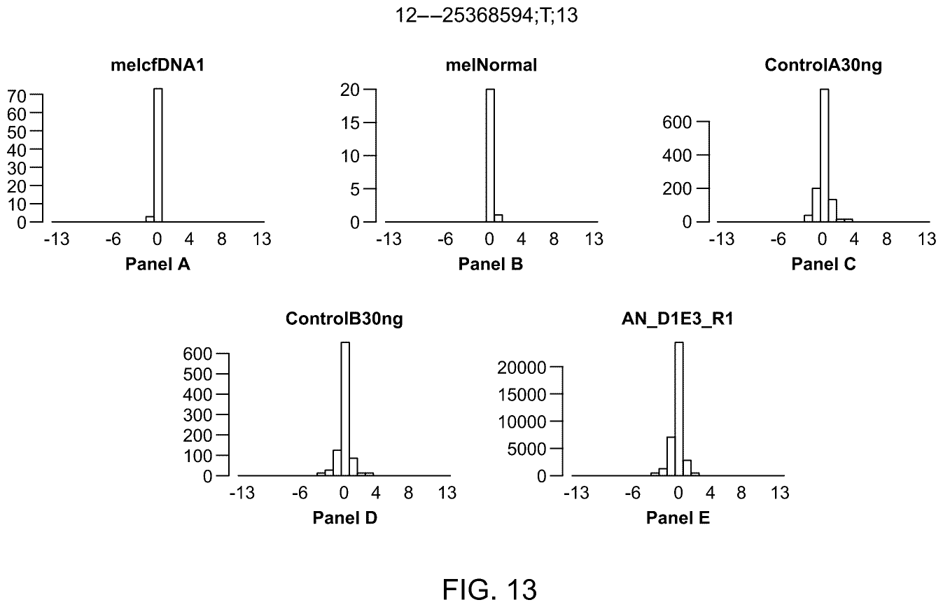

FIG. 13 shows data that exemplifies increased variance in the inferred STR repeat number after PCR-amplification and hybridization capture. The data presented are inferred STR copy number on sequencing data from four cfDNA samples and a genomic DNA sample from peripheral blood mononuclear cells (PBMCs). Panel A: PCR-free whole genome sequencing (WGS) of cfDNA from a metastatic melanoma patient; Panel B: PCR-free WGS of PBMC genomic DNA from the same metastatic melanoma patient; Panel C: SENTRYSEQ applied to healthy donor “A” DNA (30 nanograms of input DNA; Panel D: SENTRYSEQ applied to healthy donor “B” DNA (30 nanograms of input); and Panel E: a mixture of healthy donor “A” to healthy donor “B” at a ratio of 1:1000 (20 nanograms of input).

### Example 5: Analysis of cfDNA Fragment Size Distribution

Three sequencing libraries were run on a HiSeq 2500 instrument in rapid run mode across 2 flow cells. The libraries were prepared from cfDNA samples that were obtained from cancer patient ID Nos. 009, 031 and 045 prior to commencement of treatment. Seventy nanograms of extracted cfDNA were used in the SENTRYSEQ library preparation protocol (see Materials and Methods section, above). The concentrations as determined by Qubit quantification are provided in Table 3, below: Table 3: Amount of cfDNA extracted from samples.

TABLE 3
Amount of cfDNA extracted from samples.

| Patient | Cancer | Conc | Conc (ng/ml | Total DNA |
|---|---|---|---|---|
| ID No: | Type: | (ng/ul): | plasma): | extracted (ng): |
| 009 | Thoracic | 0.7 | 10.0 | 120.6 |
| 031 | Colorectal | 0.8 | 11.9 | 142.2 |
| 045 | Breast | 0.40 | 6.0 | 72 |

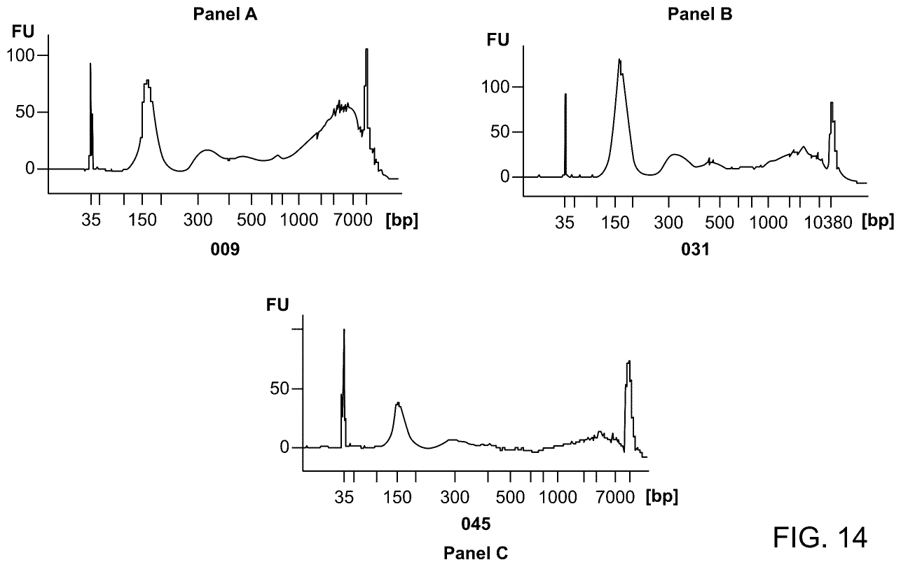

Each sample was run on a bioanalyzer instrument to determine the fragment size distribution of the cfDNA. The results are shown in FIG. 14, Panels A, B, and C, which provide bioanalyzer traces showing fragment size of extracted cfDNA in base pairs. A characteristic cfDNA peak at approximately 167 bp is present in all samples; however, the sample from patient ID No: 009 appears to have contributions from longer fragment lengths, possibly suggesting contamination from white blood cell genomic DNA.

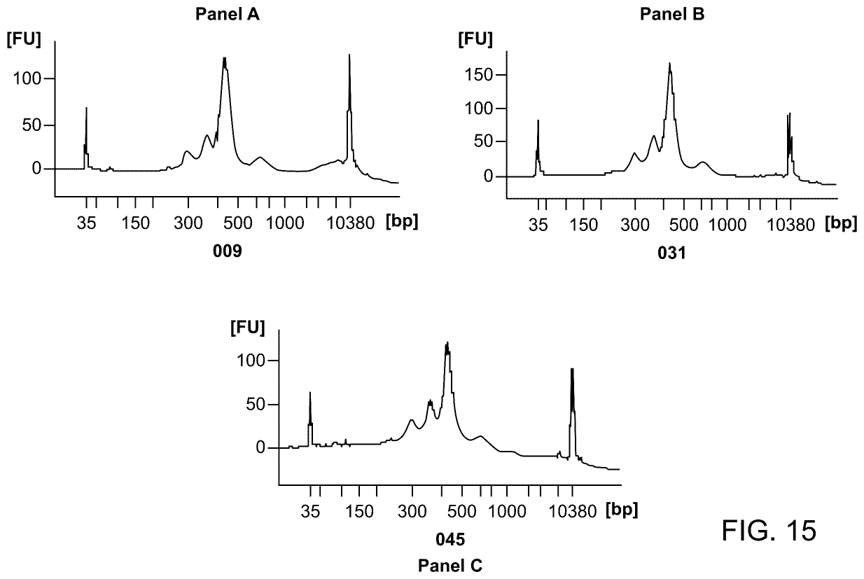

End repair and A-tailing were carried out as described in the SENTRYSEQ protocol. Ligation of adapters was carried out using a modified protocol of 16° C. for 16 hours. Samples were cleaned up using sample purification beads at a sample-to-bead ratio of 1:1.6, and then again at a ratio of 1:1. Sample was then eluted into 27.5 μL of resuspension buffer. One microliter of each sample was run on a bioanalyzer instrument to determine the fragment size distribution of the cfDNA. The results are shown in FIG. 15, Panels A, B, and C, which provide bioanalyzer traces showing library fragment size prior to PCR amplification. The observed mode of tri-modal distribution is compatible with adapter stacking (cfDNA fragment length+(4×adapter length)).

Amplification was conducted on each sample. Twenty microliters of each sample was used in an 8 cycle PCR reaction. Reaction components are provided in Table 4, below:

TABLE 4
8 cycle PCR reaction mixture components.

| Component | Volume (ul) |
|---|---|
| KAPA HiFi Mastermix | 25 |
| 10 uM Forward primer | 2.5 |
| 10 uM Reverse primer | 2.5 |
| Template DNA | 20 |

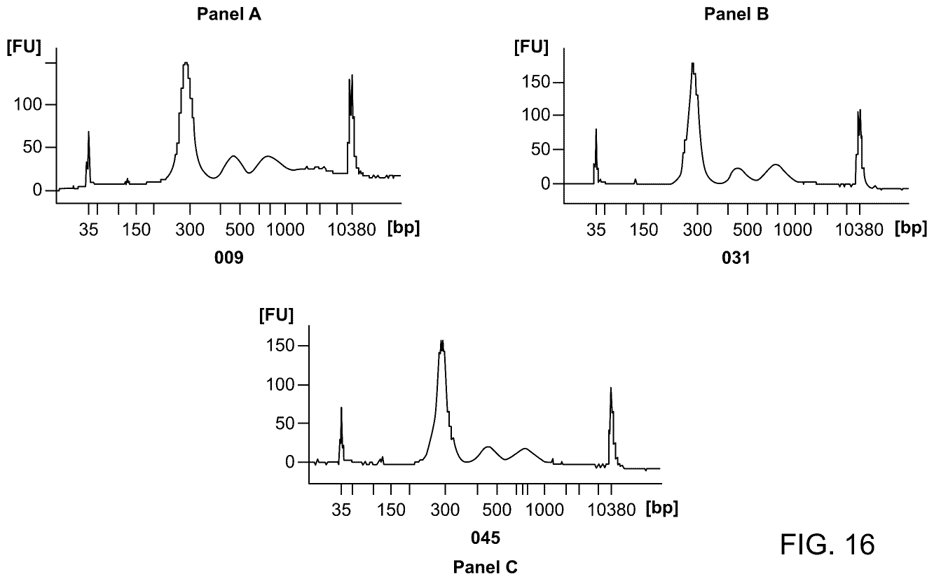

Following the PCR reaction, samples were cleaned up using sample purification beads at a ratio of 1:1.6, and eluted into a volume of 22 μL. One microliter of each sample was then run on a bioanalyzer instrument and 3 μL of each sample was used to quantify the library concentration by quantitative PCR (qPCR) in triplicate. Results are provided in FIG. 16, Panels A, B, and C, which shows library fragment sizes after 8 cycles PCR amplification. The observed mode of tri-modal distribution was shifted towards fragment length+(2×adapter length). This evidences the resolution of daisy-chained y-shaped sequencing adapters during PCR by steric hindrance, resulting in a majority population of sequencing-compatible molecules with one adapter on each end of the molecule. The concentration of each library following 8 cycles of PCR amplification is provided in Table 5, below:

TABLE 5
Library concentrations after 8 cycles of PCR amplification.

| | Concentration of library |
|---|---|
| Library name | post 8 cycle PCR (nM) |
| 009 | 397 |
| 031 | 472 |
| 045 | 449 |

Pull-down and hybridization were then performed on each library. Five hundred ng of cfDNA from each library, 50 μg of Cot-1 DNA, and 1 μL of each universal oligo were dried down in a speedvac. The contents of each tube were re-suspended in 2×7.5 μL hybridization buffer, 3 μL hybridization component, and 2.5 μL nuclease-free water. The re-suspended material was incubated in a thermocycler at 95° C. for 10 minutes, and then 3 pico moles of Lockdown probes (IDT, Iowa) was added. Two-hundred kilobases of target regions were identified using a panel selector that maximized the number of expected patient mutations within TCGA and COSMIC databases using cross-fold validation and accounting for reference sequence uniqueness. The panel optimization method identifies recurrent somatic mutations using a greedy approach. First, somatic variant calls are obtained from external and/or internal cancer genomics datasets. Second, genomic regions are weighted based on a model of predicted enrichment performance. Third, the greedy optimization identifies panel regions that maximize the total number of expected mutations in the observed data under the constraint of a certain total panel size and/or pre-specified regions or variants of interest. Fourth, the designed panel is optionally evaluated in a cross-fold validation framework to account guard against overfitting to the observed training data. These and other related techniques are described in U.S. Provisional Patent Application No. 62/286,110, the disclosure of which is herein incorporated by reference in its entirety. Lockdown probes were then ordered that covered these target regions. The hybridization mixture was incubated at 60° for 16 hours, and an IDT protocol was then used to bind target to streptavidin beads and wash away unbound target. For each sample, an aliquot was taken and quantified by qPCR. The results are provided in Table 6, below:

TABLE 6
Library concentrations post pull-down.

| | Concentration of library |
|---|---|
| Library name | pre 12 cycle PCR (pM) |
| 009 | 0.7 |
| 031 | 1.6 |
| 045 | 1.2 |

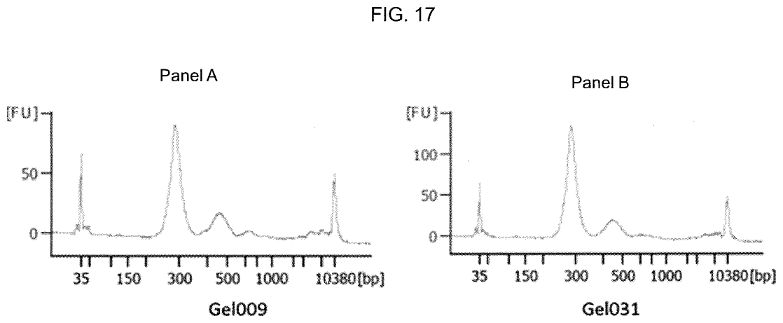

Twelve cycles of PCR were then conducted on a 20 μL sample from each library. Clean-up was carried out with Agencourt ampure XP beads according to standard procedure. The final library was then eluted into 22 μL of IDTE, and samples from each library were run on a bioanalyzer to determine the cfDNA fragment size distribution. The samples were also quantified using qPCR in triplicate. The results are provided in FIG. 17, Panels A & B, which show the cfDNA fragment size distribution for the 009 and 031 libraries. The final library concentrations, as determined by qPCR, are shown in Table 7, below:

TABLE 7
Final library concentrations.

| | Conc of library |
|---|---|
| Library name | post 12 cycle PCR (nM) |
| 009 | 4.1 |
| 031 | 5.6 |
| 045 | 3.9 |

Sequencing was conducted on each library sample. Samples were diluted to 2 nM initially, and then to a final concentration of 13.5 pM. 600 μL of each sample was then loaded into a HiSeq instrument. Desired cluster generation was 850-1000 K/mm² on a rapid run. Observed cluster generation was very low at 120 K/mm² on both flow cells, resulting in the run being aborted. The run was repeated using one flow cell, and using 600 μL of sample at a concentration of 20 pM. From this run, observed cluster generation was 1031 K/mm² and 926 K/mm² on lanes 1 and 2, respectively. From these results, it was determined that 600 μL of sample at a concentration of 20 pM in the HiSeq instrument in rapid run mode provided optimal results.

### Example 6: Identification of Cancer Recurrence in Colorectal Cancer Patients Undergoing Surgical Resection with Curative Intent

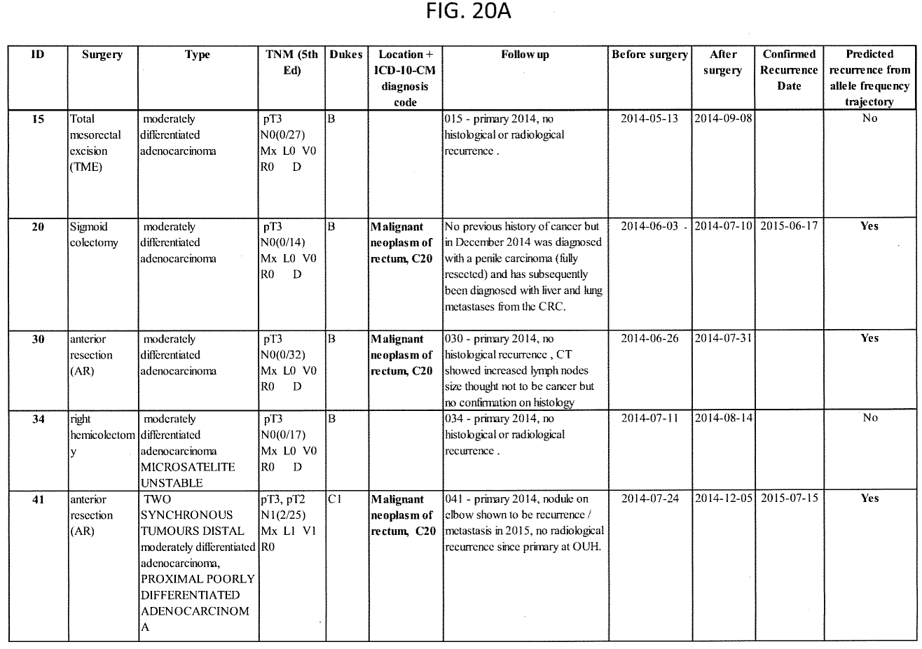

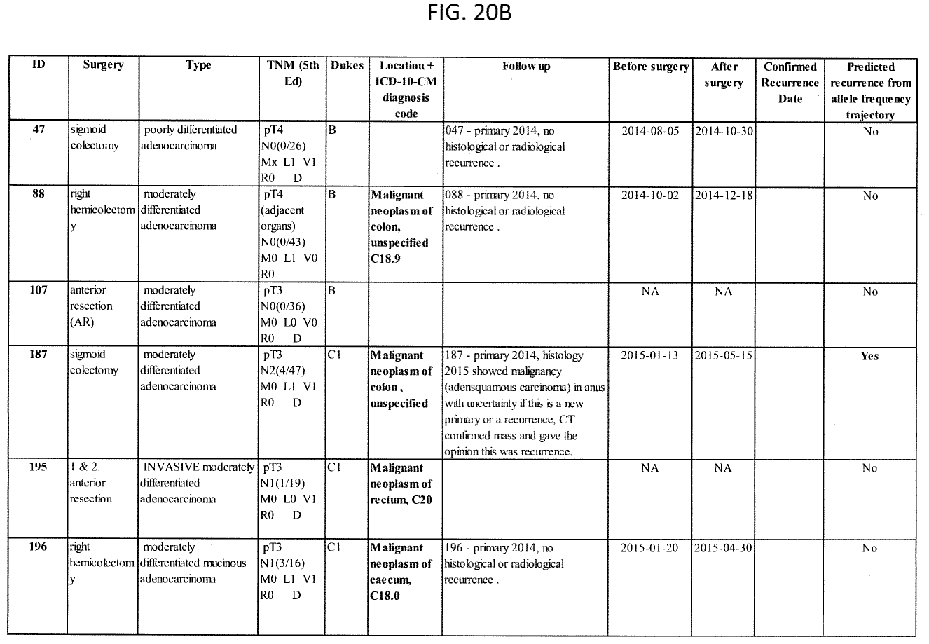

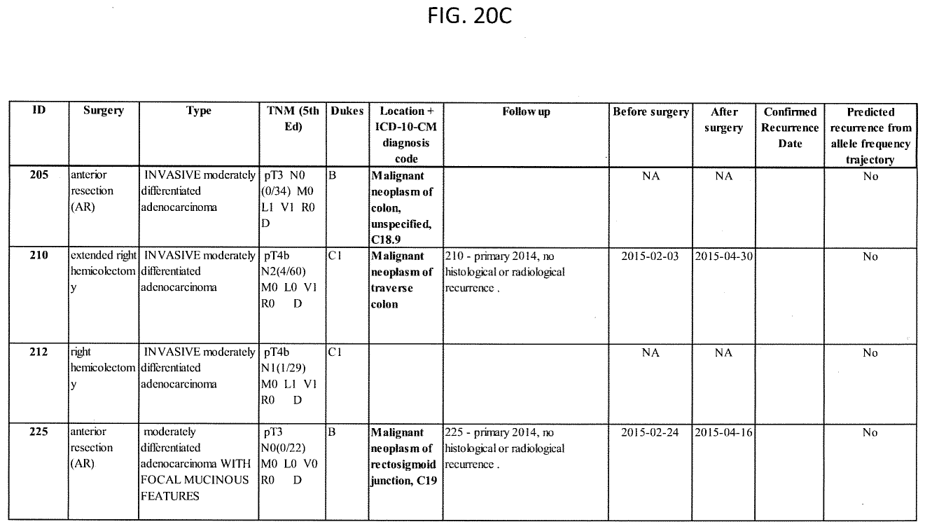

Clinical information was collected for fifteen colorectal cancer patients that underwent surgical resection with curative intent. Three patients had clinically confirmed recurrence within the study period. Ten patients were found to have metastatic cancer after surgery. Somatic trajectory tracking was used to identify both confirmed recurrence cases and two additional predicted cancer recurrences. Patient information and predicted recurrence from cfDNA analysis are provided in FIGS. 20A-C. The results demonstrate that somatic trajectory tracking in accordance with methods of the invention can be used to detect disease recurrence and/or MRD.

### Example 7: Genome-Wide cfDNA Sequencing of Melanoma Progression

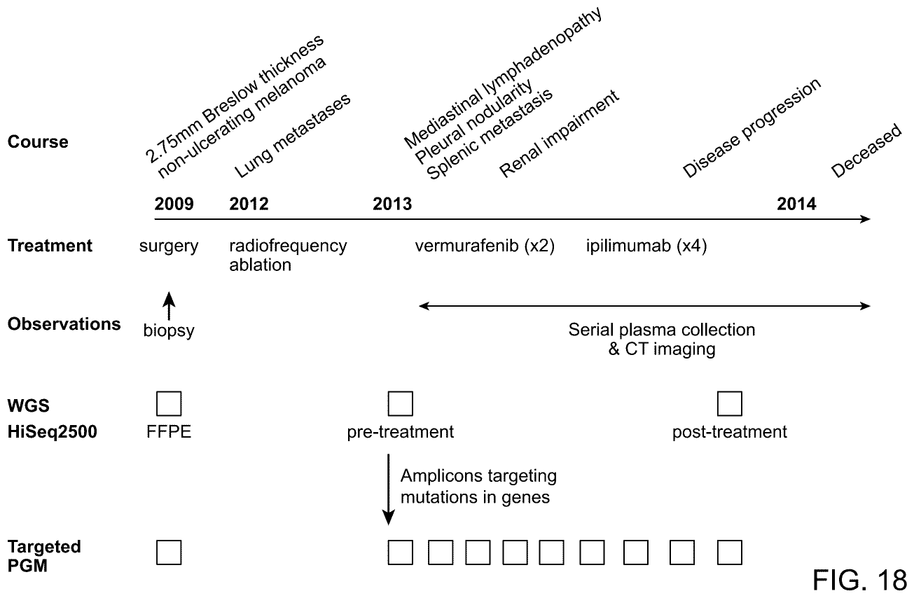

A single metastatic melanoma patient was tracked over the course of disease progression. A biopsy of the tumor was conducted early in the disease progression, and formalin-fixed paraffin-embedded (FFPE) samples were prepared for analysis. Serial plasma collection and CT imaging was conducting as the disease progressed. FIG. 18 illustrates a time course of disease progression for the patient, and shows treatment, observations and sample collection time points.

Samples were analyzed to compare the probative value of cfDNA and FFPE samples. The results are provided in FIG. 2. FFPE blocks are widely used, preserving tissue morphology but damaging nucleic acids. The most common artifacts are C>T base substitutions caused by deamination of cytosine bases, and strand breaks. C>T base substitutions induce false signals of somatic point mutations, while deamination of cytosine bases increases variance in the genome-wide coverage of template molecules. The results of this study indicate that coverage uniformity is severely impacted by FFPE sample preparation, as compared to cfDNA analysis. For example, FIG. 2 shows that cfDNA WGS has much superior sequencing uniformity compared to FFPE WGS. If coverage was perfectly uniform across the genome, traces would track the diagonal. Deviation from the diagonal indicates non-uniformity.

FIG. 3 provides a dynamic melanoma mutation signature obtained by cfDNA WGS. WGS-identified mutations stratified by triplet context (Ntimepoint1=24377, Ntimepoint2=35036) are shown. The observed profile is concordant with a Type 2 profile reported by Alexandrov et al., (2013) Signatures of mutational processes in human cancer, Nature 500.7463 (2013): 415-421, compatible with UV-induced DNA damage (abundant C>T). The first and second panels show mutational cfDNA WGS (bootstrapping, 95% CI). The third panel shows relative change in frequency between time points, stars represent significant changes (p<0.05, FET). FIG. 3 illustrates an example of a time-based progression of a melanoma mutation signature.

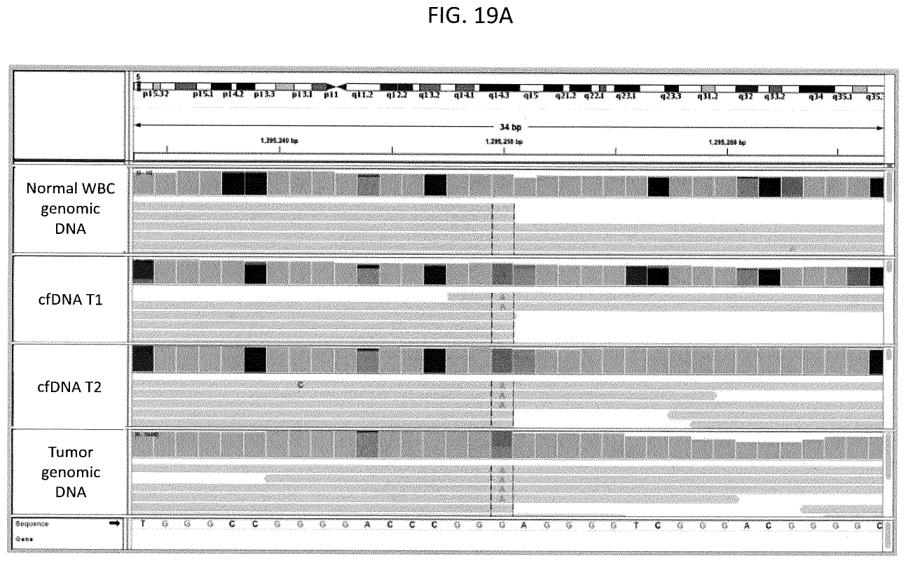

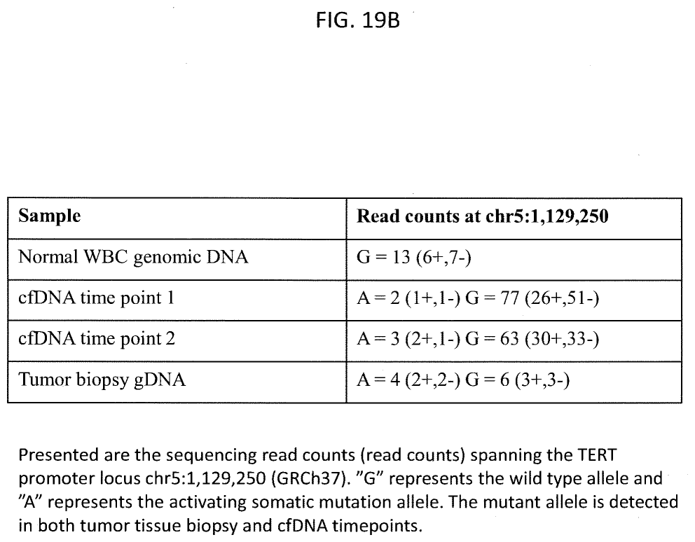

FIG. 19 illustrates transcription activating C>T mutation in the core promoter of telomerase reverse transcriptase (TERT). The mutation generates a consensus binding site for ETS transcription factors, resulting in a 2-4 fold increased transcription versus wild-type promoter status as reported by Huang et al., (2013), Highly recurrent TERT promoter mutations in human melanoma, Science 339.6122 (2013): 957-959.

FIGS. 5A-K illustrate the allele frequency trajectories of 100 somatic mutations over a course of treatment. Variants were tracked by amplicon-based sequencing of cfDNA samples on PGM (Life Tech). Loci were assigned one of 8 clusters based on hierarchical clustering (Euclidean distance) of variant allele frequency (VAF) trajectories measured across 10 time points. Alternative time series clustering approaches known in the art can be used to cluster VAF trajectories and optionally including functional annotation of variants in the clustering procedure. Treatment cycles of vemurafenib (first two rectangles in the “Treatment” row, located above the x-axis) and ipilimumab (third rectangle in the “Treatment” row, located above the x-axis) are indicated in FIGS. 5A-K. Also shown are the tumor diameters for prevascular lymph node (“Prevascular LN” row, located above the x-axis) and paratracheal lymph node (“Paratracheal LN” row, located above the x-axis) obtained using CT imaging.

FIG. 5A shows all 100 variants plotted together on the same chart.

FIG. 5B shows 54 somatic mutations.

FIG. 5C shows 1 somatic mutation (Clorf43).

FIG. 5D shows 24 somatic mutations including BRAF V600R.

FIG. 5E shows 10 somatic mutations. This population of low frequency variants do not increase with increasing tumor burden. As such, this population is interpreted to comprise non-tumor-derived somatic mutations. False positive results could also generate variants of this nature, but in illustrated example, these variants (mutations) were validated across two different sequencing technologies, thereby reducing the likelihood that they are the result of false positive results.

FIG. 5F shows 3 somatic mutations. (ADAMDEC1; CSMD1; BFSP1).

FIG. 5G shows the trajectory of a single somatic variant that is not associated with the trajectories of the other 100 tracked variants (BLACE). This variant does not track with other mutation trajectories under treatment. Accordingly, this variant is interpreted to be a non-tumor-derived somatic mutation.

FIG. 5H shows 4 somatic mutations. These somatic variants tend to have the highest VAF at any given time point (CSMD1; PKHDIL1; CSMD3; UNCSD).

FIG. 5I shows 2 somatic mutations (ST18; ADAM2).

FIG. 5J shows 1 somatic mutation (TRPS1).

FIG. 5K shows the VAF trajectory of a single clinically actionable somatic nonsynonymous variant BRAF V600R driver mutation. The BRAF V600R mutation is predicted to be sensitive to the BRAF inhibitor vemurafenib McArthur, Grant A., et al. “Safety and efficacy of vemurafenib in BRAF V600E and BRAF V600K mutation-positive melanoma (BRIM-3): extended follow-up of a phase 3, randomised, open-label study.” The lancet oncology 15.3 (2014): 323-332. WGS of cfDNA identified the activating mutation BRAF V600R at 6% VAF, concordant with a VAF of 5% estimated from amplicon sequencing on a PGM instrument. Under vemurafenib treatment the BRAF V600R mutation VAF drops to non-detectable levels using the amplicon sequencing approach. CT imaging shows a decrease in tumor volume during the same time period. Importantly, other tracked mutations continued at detectable levels during treatment showing the value of tracking a plurality of somatic variants to improve estimates of treatment response. This is further demonstrated in detecting lack of response to checkpoint inhibition therapy in the patient (variant BRAF V600R).

By tracking the allelic frequencies of the somatic mutations from cfDNA, it would have been possible to see early on that treatment with ipilimumab was ineffective in this patient. An increase in allelic frequencies is detectable 88 days before the third CT imaging scan. The variant allele frequency trajectory was highly correlated (86% Pearson correlation) with aggregated imaged node diameter. By tracking a plurality of allelic frequencies over time, the subject methods facilitate detection of a variety of different responses by a patient, including, but not limited to, disease progression and response to therapy. For example, as demonstrated by FIGS. 5A-K, a diminishing response to therapy was observed in the patient, and the aggregate allelic frequency of somatic mutations increased as the disease progressed. In some embodiments, a correlation is observed between tumor size and the allelic frequencies of the aggregated somatic mutations. Accordingly, in the depicted example, the subject methods facilitated monitoring of disease progression as well as response to therapy by tracking both individual mutations as well as the aggregate allelic frequency of mutations over time.

This example demonstrates that cfDNA WGS is readily applicable to patients with high system tumor burden, and enables comprehensive evaluation of clonal genomic evolution associated with treatment response and resistance. In addition, cfDNA results in more uniform WGS libraries than libraries from FFPE biopsies.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

1.-35. (canceled)

36. A non-transitory computer readable storage medium with computer executable instructions stored thereon executed by one or more processors to perform a method of tracking a longitudinal health status of a patient, over a plurality of timepoints comprising a first timepoint and at least one subsequent timepoint, the method comprising:
obtaining a plurality of samples from the patient, wherein a sample is obtained at each timepoint;
generating, using the plurality of samples, a plurality of mutation signatures for the patient at each timepoint;
generating, using the plurality of mutation signatures, a longitudinal health status of the patient over the plurality of timepoints;
analyzing the longitudinal health status of the patient by tracking changes in the longitudinal health status of the patient over the plurality of timepoints to determine treatment efficacy, observe disease progression, or a combination thereof.

37. The method of claim 36, wherein generating the plurality of mutation signatures further comprises:
creating a mutation signature for the patient at each timepoint, using the sample from the timepoint, wherein the mutation signature comprises:
detection of a set of somatic variants in the sample;
a sequence context factor for each somatic variant of the set of detected somatic variants;
an allele frequency of each somatic variant of the set of detected somatic variants; and
a variant type classification of each somatic variant of the set of detected somatic variants.

38. The method of claim 36, wherein generating the longitudinal health status further comprises:
comparing each somatic variant of the set of detected somatic variants in each mutation signature of the plurality of mutation signatures to a database of somatic variants known to have computationally inferred functional impact associated with cancer;
generating a health status of the patient at each timepoint by confirming presence or absence of cancer in the patient at the timepoint based on the comparison of somatic variants, wherein presence of a somatic variant known to have computationally inferred functional impact associated with cancer in the mutation signature indicates a presence of cancer in the patient at the timepoint, and wherein absence of any somatic variants known to have computationally inferred functional impact associated with cancer in the mutation signature indicates an absence of cancer in the patient at the timepoint; and
generating a longitudinal health status of the patient over the plurality of timepoints using the health status of the patient at each timepoint of the plurality of timepoints.

39. The method of claim 36, wherein analyzing the longitudinal health status of the patient by tracking changes in the longitudinal health status of the patient over the plurality of timepoints comprises:
identifying identical somatic variants in the set of detected somatic variants at two or more timepoints of the plurality of timepoints;
analyzing changes in the allele frequency of each of the identified identical somatic variants,
wherein a decrease in the allele frequency of each of the identified somatic variants from detectable levels at a timepoint of the plurality of timepoints to non-detectable levels in a later timepoint of the plurality of timepoints indicates an absence of minimum residual disease,
wherein an increase in the allele frequency of any of the identified identical somatic variants from a timepoint to a later timepoint indicates a presence of minimum residual disease, and
wherein no change in the allele frequency of any of the identified identical somatic variants from detectable levels at a timepoint to detectable levels in a later timepoint indicates a presence of minimum residual disease.

40. The method of claim 36, further comprising comparing the longitudinal health status of the patient to longitudinal health statuses contained in the one or more databases of patients with known health statuses before determining a diagnosis or therapy.

41. The method of claim 36, wherein the first timepoint is a timepoint prior to a treatment being performed or administered to the patient, and wherein the subsequent timepoint is a timepoint after the treatment is performed or administered to the patient.

42. The method of claim 36, wherein the first timepoint and subsequent timepoint are timepoints after a treatment has been performed or administered to the patient.

43. The method of claim 41, wherein the treatment comprises performance of a tumor resection surgery or administration of an anti-cancer therapeutic agent.

44. The method of claim 42, wherein the treatment comprises performance of a tumor resection surgery or administration of an anti-cancer therapeutic agent.

45. The method of claim 36, wherein the sequence context factor comprises a triplet sequence context surrounding each somatic variant, comprising three nucleotides at a position 3′ to each somatic variant and three nucleotides at a position 5′ to each somatic variant.

46. The method of claim 38, wherein the cancer is one or more of: melanoma, colorectal cancer (CRC), thoracic cancer, or breast cancer.

47. The method of claim 39, wherein the disease is a cancer selected from one or more of: melanoma, colorectal cancer (CRC), thoracic cancer, or breast cancer.

48. A system comprising:
one or more data processors; and
a non-transitory computer readable storage medium containing instructions which, when executed on the one or more data processors, cause the one or more data processors to:
obtain a plurality of samples from the patient, wherein a sample is obtained at each timepoint;
generate, using the plurality of samples, a plurality of mutation signatures for the patient at each timepoint;
generate, using the plurality of mutation signatures, a longitudinal health status of the patient over the plurality of timepoints;
analyze the longitudinal health status of the patient by tracking changes in the longitudinal health status of the patient over the plurality of timepoints to determine treatment efficacy, observe disease progression, or a combination thereof.

49. The system of claim 48, wherein generation of the plurality of mutation signatures further comprises:
creating a mutation signature for the patient at each timepoint, using the sample from the timepoint, wherein the mutation signature comprises:
detection of a set of somatic variants in the sample;
a sequence context factor for each somatic variant of the set of detected somatic variants;
an allele frequency of each somatic variant of the set of detected somatic variants; and
a variant type classification of each somatic variant of the set of detected somatic variants.

50. The system of claim 48, wherein generation of the longitudinal health status further comprises:
comparing each somatic variant of the set of detected somatic variants in each mutation signature of the plurality of mutation signatures to a database of somatic variants known to have computationally inferred functional impact associated with cancer;
generating a health status of the patient at each timepoint by confirming presence or absence of cancer in the patient at the timepoint based on the comparison of somatic variants, wherein presence of a somatic variant known to have computationally inferred functional impact associated with cancer in the mutation signature indicates a presence of cancer in the patient at the timepoint, and wherein absence of any somatic variants known to have computationally inferred functional impact associated with cancer in the mutation signature indicates an absence of cancer in the patient at the timepoint; and
generating a longitudinal health status of the patient over the plurality of timepoints using the health status of the patient at each timepoint of the plurality of timepoints.

51. The system of claim 48, wherein analysis of the longitudinal health status of the patient by tracking changes in the longitudinal health status of the patient over the plurality of timepoints comprises:
identifying identical somatic variants in the set of detected somatic variants at two or more timepoints of the plurality of timepoints;
analyzing changes in the allele frequency of each of the identified identical somatic variants,
wherein a decrease in the allele frequency of each of the identified somatic variants from detectable levels at a timepoint of the plurality of timepoints to non-detectable levels in a later timepoint of the plurality of timepoints indicates an absence of minimum residual disease,
wherein an increase in the allele frequency of any of the identified identical somatic variants from a timepoint to a later timepoint indicates a presence of minimum residual disease, and
wherein no change in the allele frequency of any of the identified identical somatic variants from detectable levels at a timepoint to detectable levels in a later timepoint indicates a presence of minimum residual disease.

52. The system of claim 48, further comprising comparing the longitudinal health status of the patient to longitudinal health statuses contained in the one or more databases of patients with known health statuses before determining a diagnosis or therapy.

53. The system of claim 48, wherein the first timepoint is a timepoint prior to a treatment being performed or administered to the patient, and wherein the subsequent timepoint is a timepoint after the treatment is performed or administered to the patient.

54. The system of claim 48, wherein the first timepoint and subsequent timepoint are timepoints after a treatment has been performed or administered to the patient.

55. The system of claim 53, wherein the treatment comprises performance of a tumor resection surgery or administration of an anti-cancer therapeutic agent.

56. The system of claim 54, wherein the treatment comprises performance of a tumor resection surgery or administration of an anti-cancer therapeutic agent.

57. The system of claim 48, wherein the sequence context factor comprises a triplet sequence context surrounding each somatic variant, comprising three nucleotides at a position 3′ to each somatic variant and three nucleotides at a position 5′ to each somatic variant.

58. The system of claim 50, wherein the cancer is one or more of: melanoma, colorectal cancer (CRC), thoracic cancer, or breast cancer.

59. The system of claim 51, wherein the disease is a cancer selected from one or more of: melanoma, colorectal cancer (CRC), thoracic cancer, or breast cancer.
